(19) United States
(12) Patent Application Publication
(10) Pub. No.: US 2025/0368639 A1
(43) Pub. Date: Dec. 4, 2025

(54) SOLID FORMS OF A COMPOUND FOR TREATING OR PREVENTING HYPERURICEMIA OR GOUT

(71) Applicant: ATOM THERAPEUTICS CO., LTD, Hangzhou, Zhejiang (CN)

(72) Inventors: Dongfang SHI, Fremont, CA (US); Changjin FU, Hangzhou (CN); Yan YANG, Hangzhou (CN)

(21) Appl. No.: 18/867,411

(22) PCT Filed: May 19, 2023
(86) PCT No.: PCT/CN2023/095342

§ 371 Date: Nov. 19, 2024

(30) Foreign Application Priority Data
May 20, 2022 (WO) PCT/CN2022/094043

(51) Int. Cl. C07D 471/04; A61K 31/437

(52) U.S. Cl. CPC C07D 471/04; A61K 31/437

(57) ABSTRACT

Crystalline forms of Compound I, active on URAT1, were prepared and characterized: Also provided are methods of using the crystalline forms.

### CROSS REFERENCE TO RELATED APPLICATIONS

This application claims the benefit of International Patent Application Number PCT/CN2022/094043, filed on May 20, 2022, which is incorporated by reference herein by reference in its entirety.

### FIELD

The present disclosure relates generally to crystalline forms of Compound I, named 3-bromo-5-[(2-ethylimidazo[1,2-a]pyridin-3-yl) carbonyl]-2-hydroxybenzonitrile, or of a pharmaceutically acceptable salt or solvate thereof; processes for making the crystalline forms; and their therapeutic methods of use.

### BACKGROUND

There remains a need to develop effective treatments for subjects suffering from or at risk of hyperuricemia and gout. Suitable compounds, including Compound I, for the treatment of such diseases and conditions are disclosed in U.S. Pat. No. 10,399,971, the disclosures of which is incorporated herein by reference in their entirety.

### SUMMARY

The present disclosure provides solid forms of Compound I or of a pharmaceutically acceptable salt or solvate thereof:

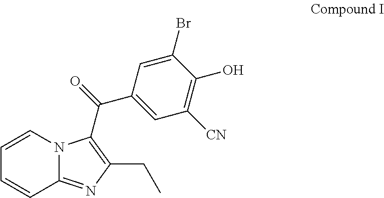 [SMILES: CCc1nc2ccccn2c1C(=O)c1cc(Br)c(O)c(C#N)c1]

The present disclosure also provides pharmaceutical compositions comprising the solid forms of Compound I. The disclosure also provides processes for making the solid forms and methods for using them in the treatment of hyperuricemia or gout.

### BRIEF DESCRIPTION OF THE DRAWINGS

FIG. 1 is the X-ray Powder Diffractogram (XRPD) of Compound I Form 1.

FIG. 2 is the Thermal Gravimetric Analysis (TGA) of Compound I Form 1.

FIG. 3 is the Differential Scanning calorimetry (DSC) of Compound I Form 1.

FIG. 4 is the XRPD of Compound I Form 2.

FIG. 5 is the TGA of Compound I Form 2.

FIG. 6 is the DSC of Compound I Form 2.

FIG. 7 is the XRPD of Compound I Form 3.

FIG. 8 is the TGA of Compound I Form 3.

FIG. 9 is the DSC of Compound I Form 3.

FIG. 10 is the XRPD of Compound I Form 4.

FIG. 11 is the TGA of Compound I Form 4.

FIG. 12 is the DSC of Compound I Form 4.

FIGS. 13A, 13B, 13C, and 13D are the XRPD of Compound I Form 5A, Form 5B, Form 5C, and Form 5D, respectively.

FIGS. 14A, 14B, 14C, and 14D are the TGA of Compound I Form 5A, Form 5B, Form 5C, and Form 5D, respectively.

FIGS. 15A, 15B, 15C, and 15D are the DSC of Compound I Form 5A, Form 5B, Form 5C, and Form 5D, respectively.

FIG. 16 is the XRPD of Compound I Form 6.

FIG. 17 is the TGA of Compound I Form 6.

FIG. 18 is the DSC of Compound I Form 6.

FIG. 19 is the XRPD of Compound I Form 7.

FIG. 20 is the TGA of Compound I Form 7.

FIG. 21 is the DSC of Compound I Form 7.

FIG. 22 is the XRPD of Compound I Form 8.

FIG. 23 is the TGA of Compound I Form 8.

FIG. 24 is the DSC of Compound I Form 8.

FIG. 25 is the XRPD of Compound I Form 9.

FIG. 26 is the TGA of Compound I Form 9.

FIG. 27 is the DSC of Compound I Form 9.

FIG. 28 is the XRPD of Compound I Form 10.

FIG. 29 is the TGA of Compound I Form 10.

FIG. 30 is the DSC of Compound I Form 10.

FIG. 31 is the XRPD of Compound I Form 11.

FIG. 32 is the TGA of Compound I Form 11.

FIG. 33 is the DSC of Compound I Form 11.

FIG. 34 is the XRPD of Compound I Form 12.

FIG. 35 is the TGA of Compound I Form 12.

FIG. 36 is the DSC of Compound I Form 12.

FIG. 37 illustrates changes in XRPD of Compound I Form 6 after solvent desorption and reabsorption.

FIG. 38 illustrates the plasma concentration-time curve of a single intravenous administration of 1 mg/kg for Compound I Form 1 in SD rats.

FIG. 39 illustrates the plasma concentration-time curve of a single intravenous administration of 5 mg/kg for Compound I Form 1 in SD rats.

FIG. 40 illustrates the plasma concentration-time curve of a single oral administration of 10 mg/kg for Compound I Form 1 in SD rats.

FIG. 41 illustrates the plasma concentration-time curve of a single oral administration of 10 mg/kg for Compound I Form 2 in SD rats.

FIG. 42 illustrates the plasma concentration-time curve of a single oral administration of 10 mg/kg for Compound I Form 5D in SD rats.

FIG. 43 illustrates a comparative X-ray powder diffraction (XRPD) plot between the Compound I Form 2 sample placed under conditions of 25° C.±2° C./60% RH±5% RH for 12 months (SPL), the Compound I Form 2 sample placed for 0 days (initial), and the Compound I Form 2 reference standard (STD).

FIG. 44 presents a comparative XRPD plot between the Compound I Form 2 sample placed under conditions of 40° C.±2° C./75% RH±5% RH for 6 months (SPL), the Compound I Form 2 sample placed for 0 days (initial), and the Compound I Form 2 reference standard (STD).

FIG. 45 shows a comparative XRPD plot between the Compound I Form 2 sample placed under conditions of 40° C.±2° C./75% RH±5% RH for 6 months (top curve) and the Compound I Form 2 reference standard (bottom curve).

FIG. 46 presents a comparative XRPD plot between the Compound I Form 2 sample placed under conditions of 25° C.±2° C./60% RH±5% RH for 12 months, 24 months, 36 months, and 48 months, 0 days, and the Compound I Form 2 reference standard (STD).

### DETAILED DESCRIPTION

The compound named 3-bromo-5-[(2-ethylimidazo[1,2-a]pyridin-3-yl) carbonyl]-2-hydroxybenzonitrile (Compound I), or a pharmaceutically acceptable salt or solvate thereof, is useful in promoting uric acid excretion or in the treatment or prevention of hyperuricemia and gout. The Compound I has the following structure:

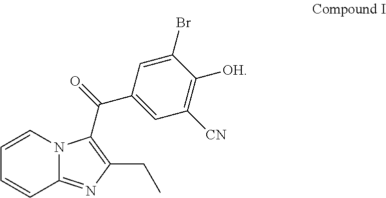 [SMILES: CCc1nc2ccccn2c1C(=O)c1cc(Br)c(O)c(C#N)c1]

The present disclosure relates to crystalline forms of Compound I or of a pharmaceutically acceptable salt or solvate thereof. The crystalline forms of Compound I or of a pharmaceutically acceptable salt or solvate thereof are described herein as “Compound I Form 1,” “Compound I Form 2. “Compound I Form 3.” “Compound I Form 4”, “Compound I Form 5A,” Compound I Form 5B,” “Compound I Form 5C,” “Compound I Form SD,” “Compound I Form 6,” “′Compound I Form 7,” “Compound I Form 8,” “Compound I Form 9,” “Compound I Form 10,” “Compound I Form 11,” and “Compound I Form 12.”

### Definitions

As used herein the following definitions apply unless clearly indicated otherwise:

All atoms designated within a Formula described herein, either within a structure provided, or within the definitions of variables related to the structure, is intended to include any isotope thereof, unless clearly indicated to the contrary. It is understood that for any given atom, the isotopes may be present essentially in ratios according to their natural occurrence, or one or more particular atoms may be enhanced with respect to one or more isotopes using synthetic methods known to one skilled in the art. Thus, hydrogen includes for example ¹H, ²H, ³H; carbon includes for example ¹¹C, ¹²C, ¹³C, ¹⁴C; oxygen includes for example ¹⁶O, ¹⁷O, ¹⁸O; nitrogen includes for example ¹³N, ¹⁴N, ¹⁵SN; sulfur includes for example ³²S, ³³s, ³⁴S, ³⁵S, ³⁶S, ³⁷S, ³⁸S; fluoro includes for example ¹⁷F, ¹⁸F, ¹⁹F; chloro includes for example ³⁵Cl, ³⁶Cl, ³⁷Cl, ³⁸Cl, ³⁹Cl; and the like.

Certain compounds contemplated for use in accordance with the present disclosure can exist in unsolvated forms as well as solvated forms, including hydrated forms. “Hydrate” refers to a complex formed by combination of water molecules with molecules or ions of the solute. “Solvate” refers to a complex formed by combination of solvent molecules with molecules or ions of the solute. The solvent can be an organic compound, an inorganic compound, or a mixture of both. Solvate is meant to include hydrate, hemi-hydrate, channel hydrate etc. Some examples of solvents include, but are not limited to, methanol, N,N-dimethylformamide, tetrahydrofuran, dimethyl sulfoxide, and water. In general, the solvated forms are equivalent to unsolvated forms and are encompassed within the scope of the present disclosure. Certain compounds contemplated for use in accordance with the present disclosure may exist in multiple crystalline or amorphous forms. In general, all physical forms are equivalent for the uses contemplated by the present disclosure and are intended to be within the scope of the present disclosure.

As used herein, the term “solid form” refers to a type of solid-state material that includes amorphous as well as crystalline forms. The term “crystalline form” refers to polymorphs as well as solvates, hydrates, etc. The term “polymorph” refers to a particular crystal structure having particular physical properties such as X-ray diffraction, melting point, and the like.

As used herein, the terms “treat,” “treating,” “therapy,” “therapies,” and like terms refer to the administration of material, e.g., any one or more solid, crystalline, or polymorphs of Compound I as described herein in an amount effective to prevent, alleviate, or ameliorate one or more symptoms of a disease or condition, i.e., indication, and/or to prolong the survival of the subject being treated.

As used herein, the term “modulating” or “modulate” refers to an effect of altering a biological activity, especially a biological activity associated with a particular biomolecule such as human uric acid transporter 1 (hURAT1). For example, certain molecules described here modulates the activity of that biomolecule, by either increasing or decreasing the activity of the biomolecule. Such activity is typically indicated in terms of an inhibitory concentration (IC₅₀) or excitation concentration (EC₅₀) of the compound for an inhibitor or activator, respectively, with respect to, for example, an enzyme.

As used herein, the term “promoting” or “promote” refers to an effect of increasing a biological activity associated with a particular molecule such as uric acid. For example, certain molecules described here promotes the excretion (e.g. expelling as waste) of that molecule, such as uric acid.

As used herein, the term “URAT1-mediated disease or condition,” refers to a disease or condition in which the biological function of URAT1, including any mutations thereof, affects the development, course, and/or symptoms of the disease or condition, and/or in which modulation of the URAT1 alters the development, course, and/or symptoms of the disease or condition. The URAT1-mediated disease or condition includes a disease or condition for which inhibition provides a therapeutic benefit, e.g., wherein treatment with URAT1inhibitor(s), including one or more solid, crystalline, or polymorphs of Compound I as described herein, provides a therapeutic benefit to the subject suffering from or at risk of the disease or condition.

As used herein, the term “composition” refers to a pharmaceutical preparation suitable for administration to an intended subject for therapeutic purposes that contains at least one pharmaceutically active compound, including any solid form thereof. The composition may include at least one pharmaceutically acceptable component to provide an improved formulation of the compound, such as a suitable carrier or excipient.

As used herein, the term “subject” refers to a living organism that is treated with compounds as described herein, including, but not limited to, any mammal, such as a human, other primates, sports animals, animals of commercial interest such as cattle, farm animals such as horses, or pets such as dogs and cats.

The term “pharmaceutically acceptable” indicates that the indicated material does not have properties that would cause a reasonably prudent medical practitioner to avoid administration of the material to a patient, taking into consideration the disease or conditions to be treated and the respective route of administration. For example, it is commonly required that such a material be essentially sterile, e.g., for injectables. The term “pharmaceutically acceptable salt” of a given compound refers to salts that retain the biological effectiveness and properties of the given compound and which are not biologically or otherwise undesirable. “Pharmaceutically acceptable salts” or “physiologically acceptable salts” include, for example, salts with inorganic acids and salts with an organic acid. In addition, if the compounds described herein are obtained as an acid addition salt, the free base can be obtained by basifying a solution of the acid salt. Conversely, if the product is a free base, an addition salt, particularly a pharmaceutically acceptable addition salt, may be produced by dissolving the free base in a suitable organic solvent and treating the solution with an acid, in accordance with conventional procedures for preparing acid addition salts from base compounds. Those skilled in the art will recognize various synthetic methodologies that may be used to prepare nontoxic pharmaceutically acceptable addition salts. Pharmaceutically acceptable acid addition salts may be prepared from inorganic and organic acids. Salts derived from inorganic acids include, e.g., hydrochloric acid, hydrobromic acid, sulfuric acid, nitric acid, phosphoric acid and the like. Salts derived from organic acids include, e.g., acetic acid, propionic acid, gluconic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluene-sulfonic acid, salicylic acid and the like. Likewise, pharmaceutically acceptable base addition salts can be prepared from inorganic and organic bases. Salts derived from inorganic bases include, by way of example only, sodium, potassium, lithium, aluminum, ammonium, calcium and magnesium salts. Salts derived from organic bases include, but are not limited to, salts of primary, secondary and tertiary amines, such as alkyl amines (i.e., NH₂(alkyl)), dialkyl amines (i.e., HN(alkyl)₂), trialkyl amines (i.e., N (alkyl)₃), substituted alkyl amines (i.e., NH₂(substituted alkyl)), di(substituted alkyl)amines (i.e., HN(substituted alkyl)₂), tri (substituted alkyl)amines (i.e., N (substituted alkyl)₃), alkenyl amines (i.e., NH₂(alkenyl)), dialkenyl amines (i.e., HN(alkenyl)₂), trialkenyl amines (i.e., N (alkenyl)₃), substituted alkenyl amines (i.e., NH₂(substituted alkenyl)), di(substituted alkenyl)amines (i.e., HN(substituted alkenyl)₂), tri (substituted alkenyl)amines (i.e., N (substituted alkenyl)₃, mono-, di- or tri-cycloalkyl amines (i.e., NH₂(cycloalkyl), HN(cycloalkyl)₂, N (cycloalkyl)₃), mono-, di- or tri-arylamines (i.e., NH₂(aryl), HN(aryl)₂, N(aryl)₃) or mixed amines, etc. Specific examples of suitable amines include, by way of example only, isopropylamine, trimethyl amine, diethyl amine, tri (iso-propyl) amine, tri (n-propyl)amine, ethanolamine, 2-dimethylaminoethanol, piperazine, piperidine, morpholine, N-ethylpiperidine and the like.

In the present context, the term “therapeutically effective” or “effective amount” indicates that the materials or amount of material is effective to prevent, alleviate, or ameliorate one or more symptoms of a disease or medical condition, and/or to prolong the survival of the subject being treated. The therapeutically effective amount will vary depending on the compound, the disorder or condition and its severity and the age, weight, etc., of the mammal to be treated. For example, an effective amount is an amount sufficient to effectuate a beneficial or desired clinical result. The effective amounts can be provided all at once in a single administration or in fractional amounts that provide the effective amount in several administrations. The precise determination of what would be considered an effective amount may be based on factors individual to each subject, including their size, age, injury, and/or disease or injury being treated, and amount of time since the injury occurred or the disease began. One skilled in the art will be able to determine the effective amount for a given subject based on these considerations which are routine in the art.

As used herein, the phrase “substantially as shown in FIG.” as applied to DSC thermograms is meant to include a variation of ±3° Celsius and as applied to TGA is meant to include a variation of ±2% in weight loss.

In the context of the use, testing, or screening of compounds that are or may be modulators, the term “contacting” means that the compound(s) are caused to be in sufficient proximity to a particular molecule, complex, cell, tissue, organism, or other specified material that potential binding interactions and/or chemical reaction between the compound and other specified material can occur.

### Crystalline Forms of Compound I

As described generally above, the present disclosure provides crystalline forms of Compound I. In some embodiments, the crystalline forms are of Compound I as a free base compound or of a solvate of the free base compound. For example, Compound I Form 1, Form 2, Form 3, and Form 4 described below are crystalline forms of Compound I as a free base compound or a solvate of the free base compound.

### Compound I Form 1

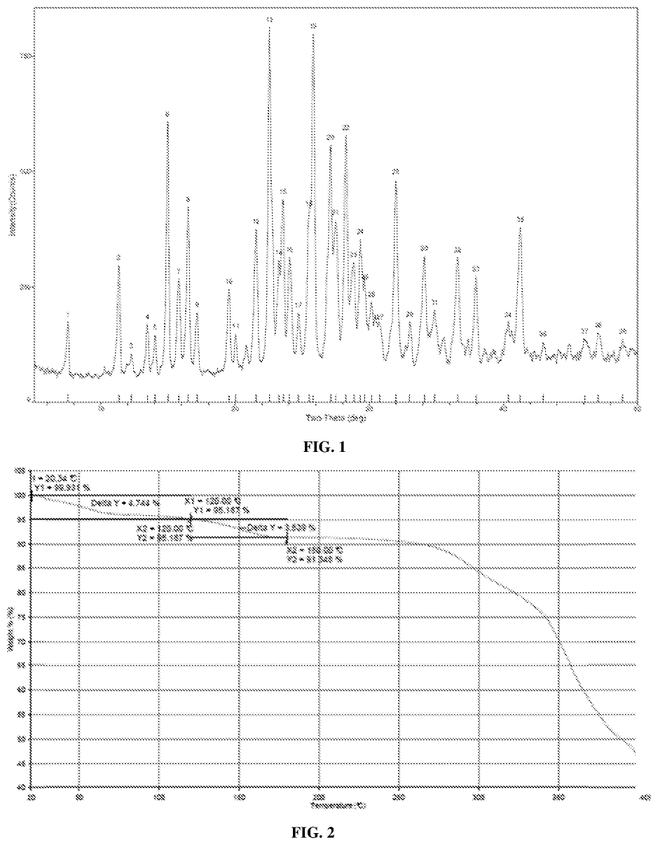

Compound I Form 1 is characterized by an XRPD comprising peaks (±0.2°) at 15.0, 22.6, 25.8, 32.0, 41.3°2θ, as determined on a diffractometer using Cu-Kα radiation. In some embodiments, the diffractogram comprises one, two, three, or four more peaks selected from (±0.2°) 25.5, 27.1, 27.5, and 28.3° 2θ. In some embodiments, the diffractogram further comprises one, two, three, four, five, six, seven, eight, nine, ten, eleven, or twelve additional peaks selected from (±0.2°) 11.3, 15.8, 16.5, 19.5,21.6, 23.3, 23.6, 28.8, 29.3, 29.7, 34.1, 36.6, and 41.3°2θ. Form 1 is also characterized by XRPD substantially as shown in FIG. 1. In one embodiment, this disclosure provides Compound I Form 1 comprising two or more peaks (±0.2°) listed herein as determined on a diffractometer using Cu-Kα radiation.

In some embodiments, Form 1 is also characterized by TGA comprising a thermogram substantially as shown in FIG. 2. As illustrated, Form 1 presents two weight loss steps. The first step is from room temperature to about 120° C., during which there is a weight loss of about 4.7%. The second weight loss is from about 120° C. to about 180° C., during which there is a weight loss of about 3.8%.

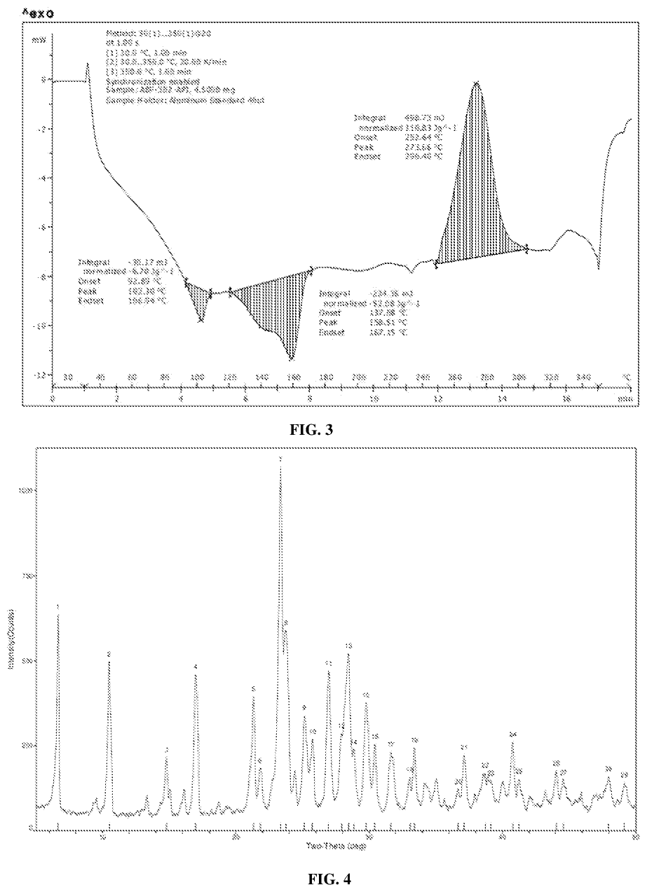

In some embodiments, Form 1 is further characterized by DSC curve substantially as shown in FIG. 3. As illustrated, Form 1 presents two endothermic peaks and one exothermic peak. The first endothermic peak has an onset at about 93° C., peaks at about 102° C., and ends at about 107° C. It is attributed to water or residual solvent escaping from the crystal. The second endothermic peak has an onset at about 138° C., peaks at about 158° C., and ends at about 167° C. It is attributed to the melting accompanied by the evaporation of crystal water. The exothermic peak has an onset at about 253° C., peaks at about 274° C., and ends at about 290° C. It is attributed to compound decomposition.

In some embodiment, Form 1 is further characterized by ¹HNMR spectrum comprising peaks at 9.2 ppm, 8.2 ppm, 8.0 ppm, 7.8 ppm, 7.3 ppm, 4.3 ppm, 2.5 ppm, and 1.2 ppm.

In some embodiments, Form 1 is a hydrate of Compound I, such as a dihydrate of Compound I

### Compound I Form 2

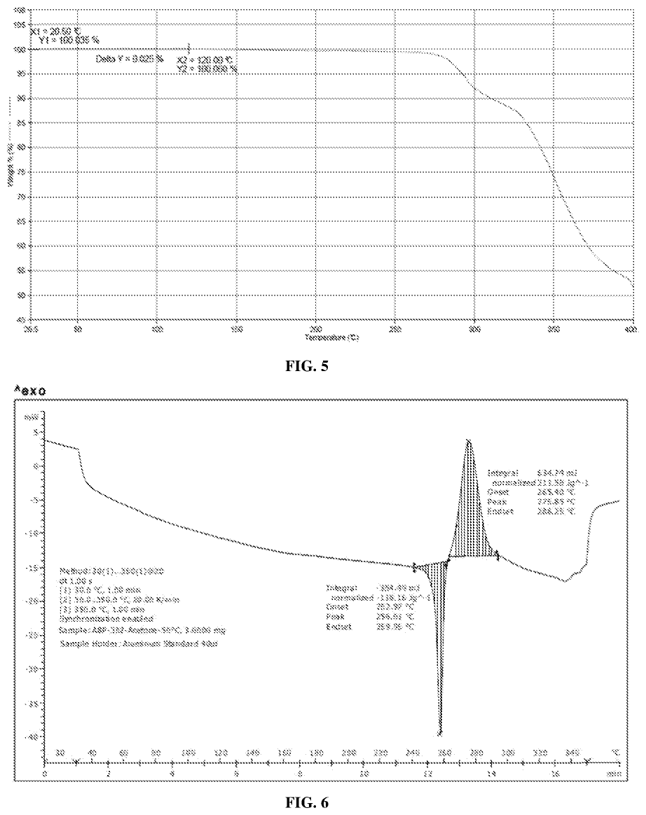

Compound I Form 2 is characterized by an X-ray powder diffractogram. The X-ray powder diffractogram comprises peaks (±0.2°) at 6.7, 10.5, 17.0, 23.4, and 26.9° 2θ, as determined on a diffractometer using Cu-Kα radiation. In some embodiments, the diffractogram comprises one, two, three, or four more peaks selected from (±0.2°) 14.8, 21.3, 28.4, and 29.8° 2θ. In some embodiments, the diffractogram comprises one, two, three, four, five, six, seven, eight, nine, ten, or eleven additional peaks selected from (±0.2°) 23.8, 25.1, 25.7, 27.9, 30.4, 40.8, 33.4, 31.6, 28.9, 37.1, and 21.8°2θ. Form 2 is also characterized by XRPD substantially as shown in FIG. 4. In one embodiment, this disclosure provides Compound I Form 2comprising two or more peaks (±0.2°) listed herein as determined on a diffractometer using Cu-Kα radiation.

In some embodiments, Form 2 is also characterized by TGA comprising a thermogram substantially as shown in FIG. 5. As illustrated, Form 2 presents no significant weight loss below about 250° C. For example, the weight loss from room temperature to about 120° C. is only about 0.03%.

In some embodiments, Form 2 is further characterized by DSC curve substantially as shown in FIG. 6. As illustrated, Form 2 presents one endothermic peaks and one exothermic peak. The endothermic peak has an onset at about 253° C., peaks at about 256° C., and ends at about 260° C. The endothermic peak is attributed to the melting of Form 2.

In some embodiments, Form 2 is an anhydrous form of Compound I.

### Compound I Form 3

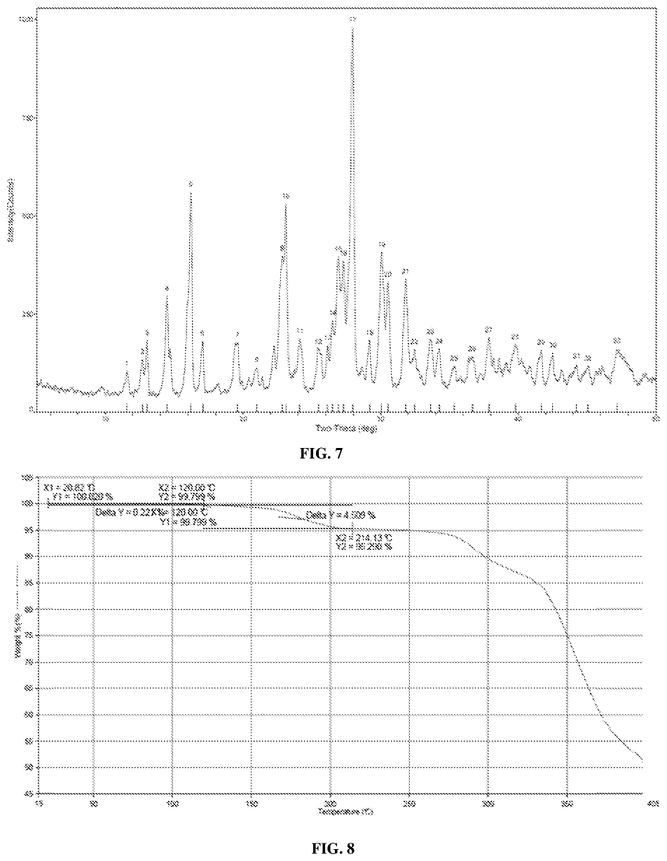

Compound I Form 3 is characterized by an X-ray powder diffractogram comprising peaks (±0.2°) at 16.2, 23.1, 28.0, and 31.8° 2θ, as determined on a diffractometer using Cu-Kα radiation. The diffractogram comprises one, two, three, four, five, six, seven, eight, nine, ten, or eleven additional peaks (±0.2°) at 13.0, 14.5, 17.1, 19.6, 22.8, 24.1, 26.5, 26.9, 27.3, 30.1, and 30.5° 2θ. Form 3 is also characterized by XRPD substantially as shown in FIG. 7. In one embodiment, this disclosure provides Compound I Form 3 comprising two or more peaks (±0.2°) listed herein as determined on a diffractometer using Cu-Kα radiation.

In some embodiments, Form 3 is also characterized by TGA comprising a thermogram substantially as shown in FIG. 8. As illustrated, Form 3 presents a weight loss of about 4.5% from about 120.0° C. to about 214° C.

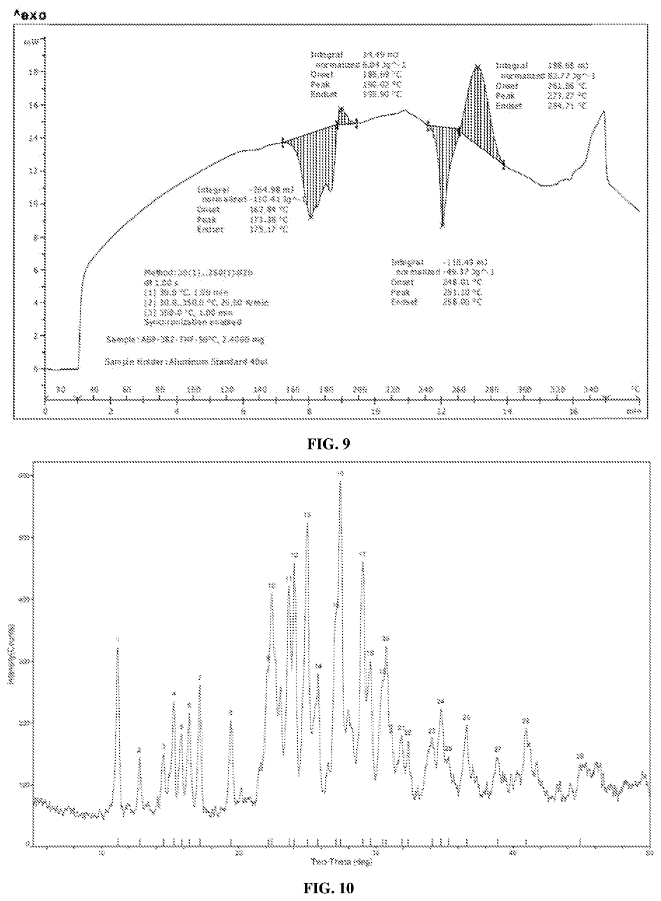

In some embodiments, Form 3 is further characterized by DSC curve substantially as shown in FIG. 9. As illustrated, Form 3 presents two endothermic peaks. The first endothermic peak has an onset at about 163° C., peaks at about 171° C., and ends at about 175° C. The peak is attributed to the solvent escaping from the crystal. The second endothermic peak has an onset at about 248° C., peaks at about 251° C., and ends at about 258° C. This peak is attributed to the melting of Form 3. There is also an exothermic peak after the first endothermic peak. It is attributable to crystal transition. In some embodiments, Form 3 is a solvate. In some embodiments, Form 3 is a monohydrate.

### Compound I Form 4

Compound I Form 4 is characterized by an XRPD comprising peaks (±0.2°) at 11.2, 22.4, 25.0, 27.4, and 29.1°2θ, as determined on a diffractometer using Cu-Kα radiation. In some embodiments, the diffractogram comprises one, two, three, four, five, or six more peaks selected from (±0.2°) 17.2, 22.2, 23.7, 24.1, 27.1, and 30.7° 2θ. In some embodiments, the diffractogram comprises one, two, three, four, five, six, seven, eight, nine, ten, eleven, or twelve additional peaks (±0.2°) at 12.8, 14.5, 15.3, 15.8, 16.4, 19.4, 25.8, 29.6, 30.5, 34.8, 36.6, and 41.0° 2θ. Form 4 is also characterized by XRPD substantially as shown in FIG. 10. In one embodiment, this disclosure provides Compound I Form 4 comprising two or more peaks (±0.2°) listed herein as determined on a diffractometer using Cu-Kα radiation.

In some embodiments, Form 4 is also characterized by TGA comprising a thermogram substantially as shown in FIG. 11. As illustrated, Form 4 presents two weight loss steps. The first weight loss step is from room temperature to about 120° C., during which there is about 2.4% weight loss. The second weight loss step is from about 120° C.′ to about 172° C., during which there is about 5.8% weight loss.

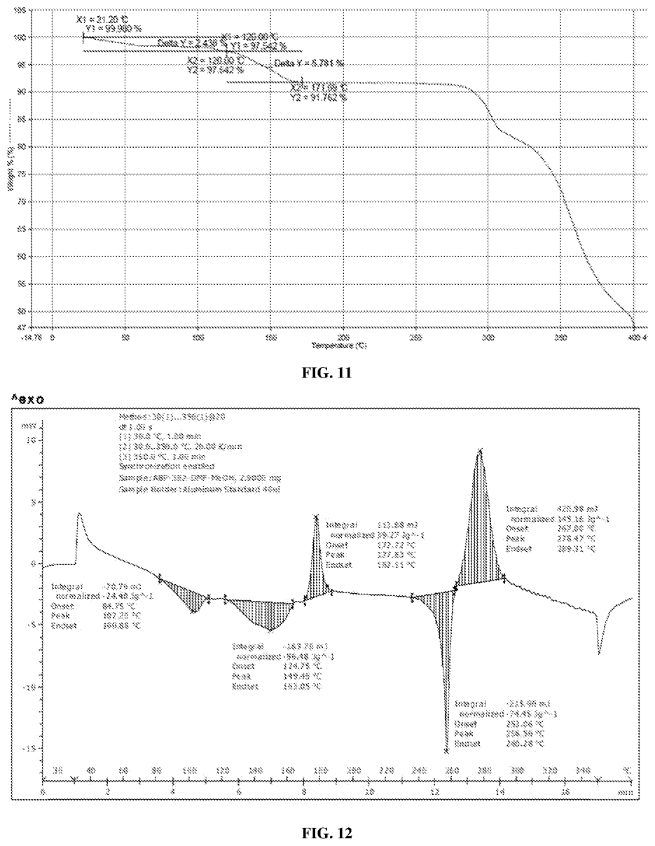

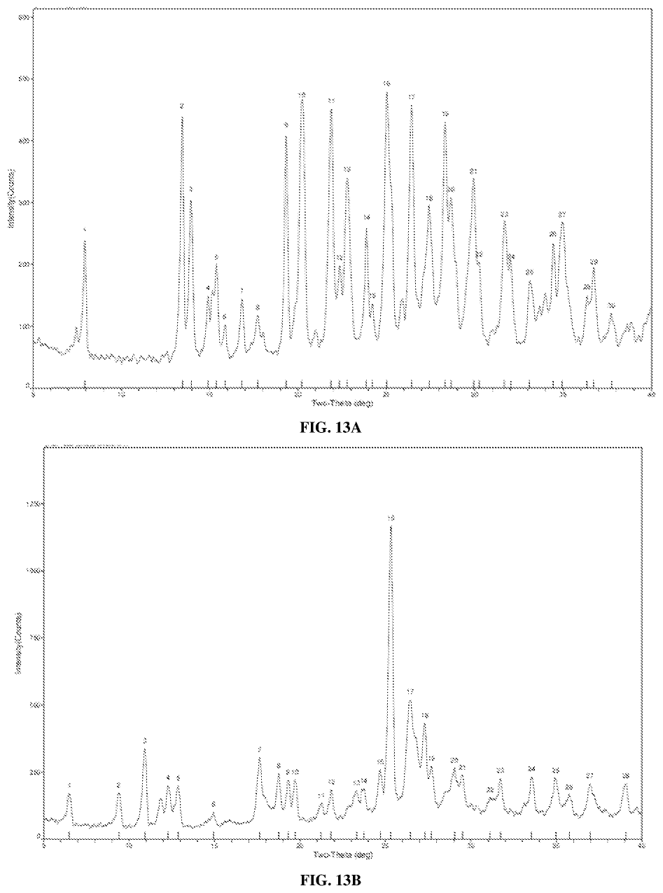

In some embodiments, Form 4 is further characterized by DSC curve substantially as shown in FIG. 12. As illustrated, Form 4 presents three endothermic peaks. The first endothermic peak has an onset at about 85° C., peaks at about 102° C., and ends at about 110° C. The second endothermic peak has an onset at about 125° C., peaks at about 149° C. and ends at about 163° C. Both these endothermic peaks are attributed to solvent escaping from the crystal. The third endothermic peak has an onset at about 253° C. peaks at about 256° C.′, and ends at about 260° C. It is attributed to the melting of Form 4. There are two exothermic peaks. The first exothermic peak has an onset at about 173° C., peaks at about 178° C., and ends at about 182° C. It is attributed to crystal transition. The second exothermic peak has an onset at about 267° C.′, peaks at about 278° C., and ends at about 289° C. It is attributed to the decomposition of Form 4. In some embodiments, Form 4 is an unstable solvate.

### Crystalline Forms of Salts of Compound I

Compound I may form a salt with suitable acids. For example, Compound I may form a salt with hydrochloric acid (referred to as the hydrochloride salt), methanesulfonic acid (referred to as the mesylate salt), benzenesulfonic acid (referred to as the besylate salt), sulfuric acid (referred to as the sulfate salt), nitric acid (referred to as the nitrate salt), or maleic acid (referred to as the maleate salt). Moreover, Compound I may form a salt with suitable bases. For example, Compound I may form a salt with potassium hydroxide (referred to as the potassium salt) or sodium hydroxide (referred to as the sodium salt). In some embodiments, provided herein are solid forms or crystalline forms of such salts of Compound I.

### Compound I Form 5A

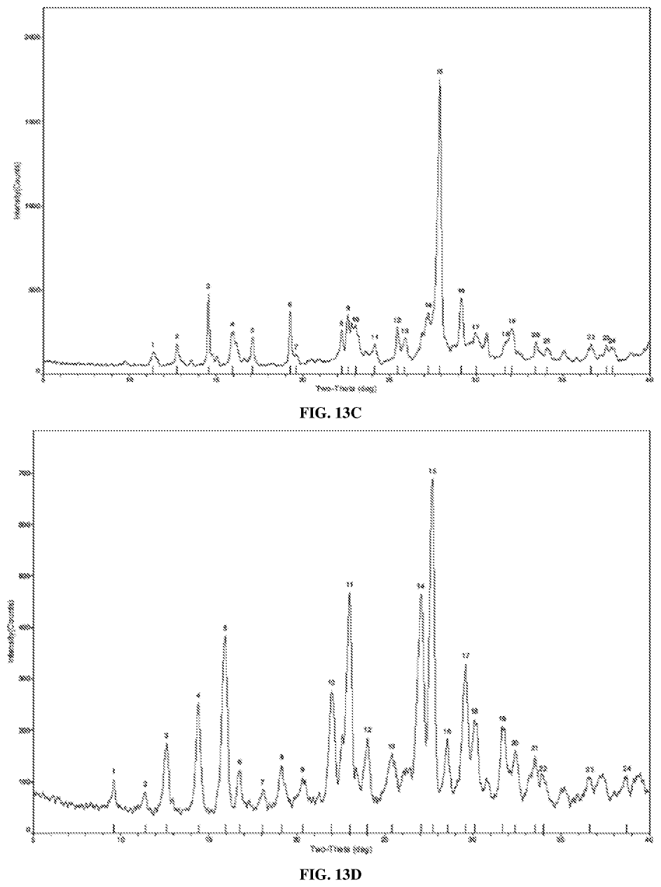

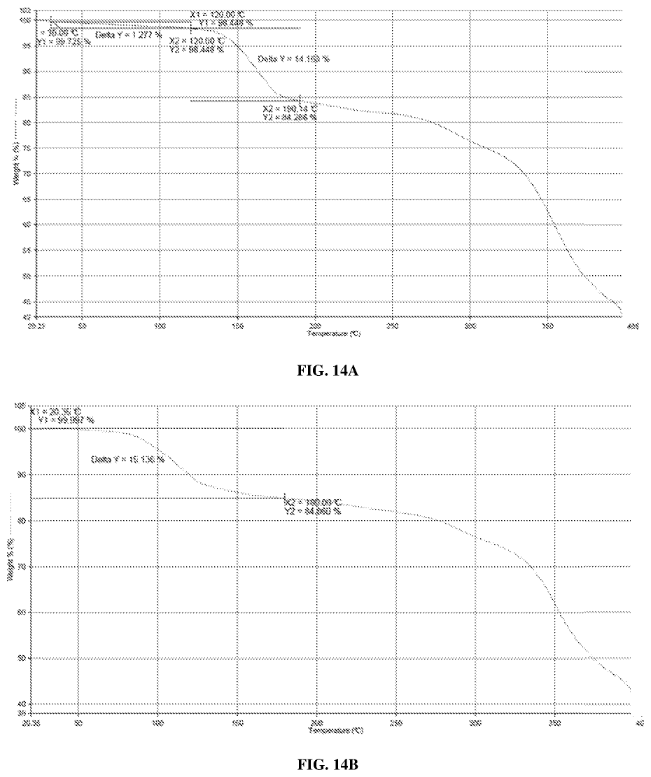

Compound I Form 5A is a crystalline form of a tetrahydrofuran solvate of the hydrochloride salt of Compound I. Form 5A is characterized by an XRPD comprising peaks (±0.2°) at 13.5, 19.3, 20.2, 21.9, 25.0, 26.4, and 27.4° 2θ, as determined on a diffractometer using Cu-Kα radiation. In some embodiments, the diffractogram comprises one, two, three, four, five, six, seven, eight, nine, or ten more peaks selected from (±0.2°) 8.0, 14.0, 22.8, 23.9, 28.4, 28.7, 30.0, 31.7, 35.0, and 36.7. In some embodiments, the diffractogram comprises one, two, three, four, five, six, seven, eight, nine, ten, eleven, twelve, or thirteen additional peaks selected from (±0.2° 14.9, 15.4, 15.9, 16.8, 17.7, 22.3, 24.2, 30.3, 32.1, 33.1, 34.5, 36.4, and 37.8° 2θ. Form 5A is also characterized by XRPD substantially as shown in FIG. 13A. In one embodiment, this disclosure provides Compound I Form 5A comprising two or more peaks (±0.2°) listed herein as determined on a diffractometer using Cu-Kα radiation.

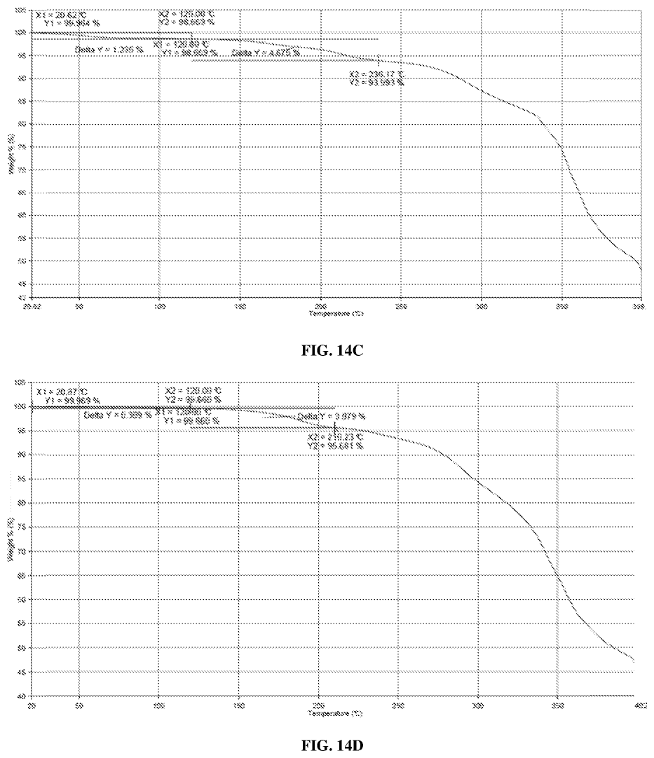

In some embodiments, Form 5A is also characterized by TGA comprising a thermogram substantially as shown in FIG. 14A. As illustrated, Form 5A presents two weight loss steps. The first step is from room temperature to about 120° C., during which there is a weight loss of about 1.3%. The second weight loss is from 120° C. to 190° C. during which there is a weight loss of about 14.2%.

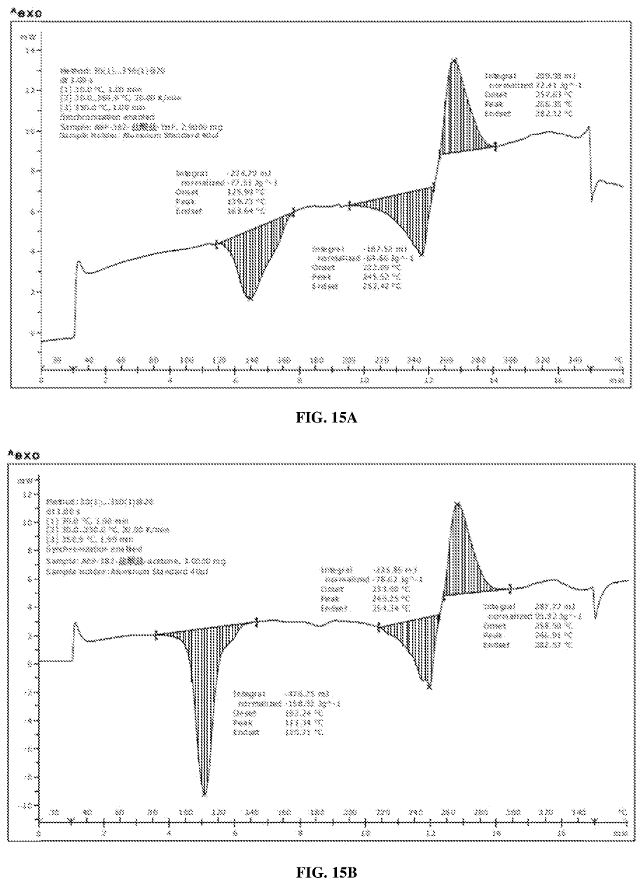

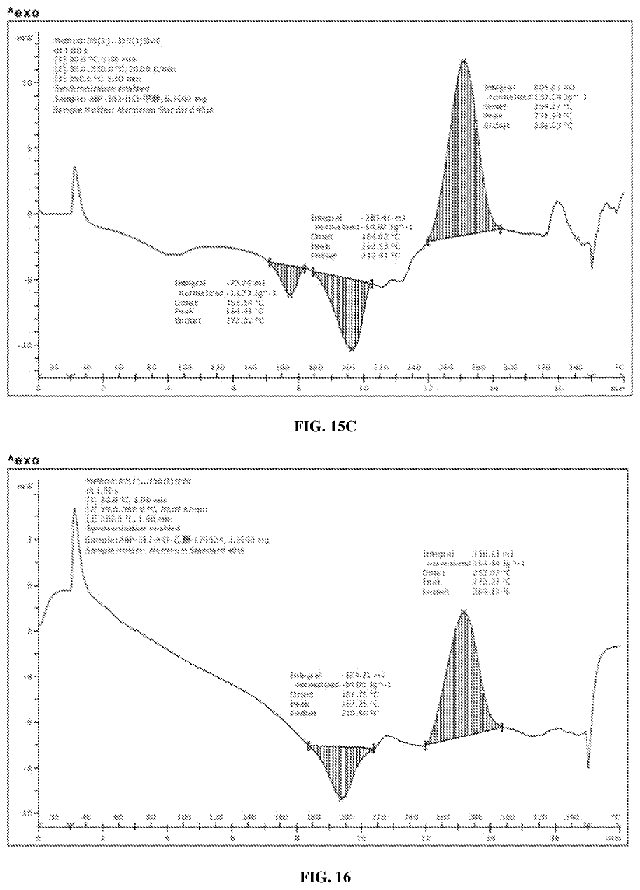

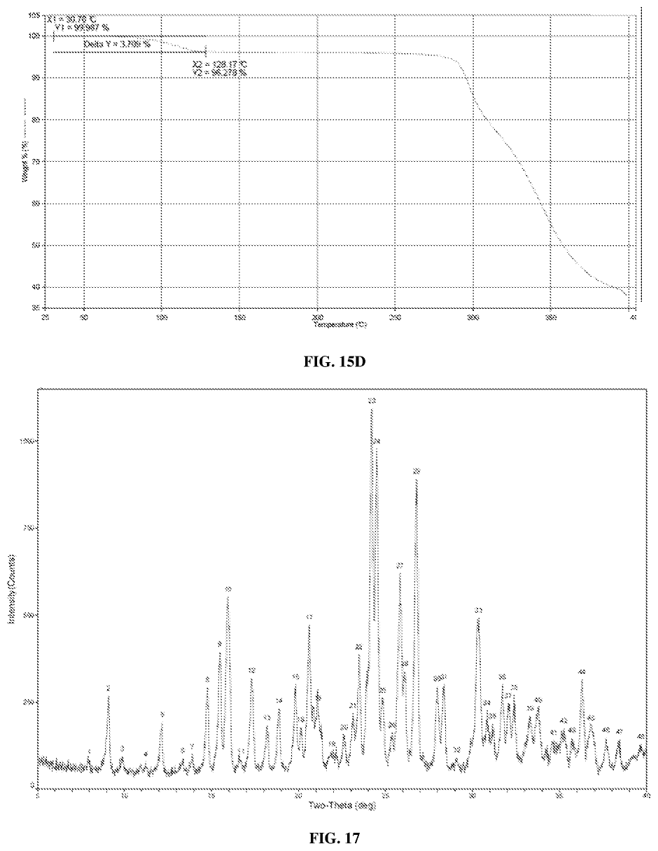

In some embodiments, Form 5A is further characterized by DSC curve substantially as shown in FIG. 15A. As illustrated, Form 5A presents two endothermic peaks and one exothermic peak. The first endothermic peak has an onset at about 126° C., a peak at about 140° C., and an endset at about 164° C. The second endothermic peak has an onset at about 222° C., a peak at about 246° C., and an endset at about 252° C. Itis attributed to the melting. The exothermic peak has an onset at about 258° C.′, a peak at about 266° C., and an endset at about 282° C. It is attributed to compound decomposition.

In some embodiment, Form 5A is further characterized by ¹HNMR spectrum comprising peaks at 9.1 ppm, 8.2 ppm, 8.1 ppm, 8.0 ppm, 7.5 ppm, 3.6 ppm, 2.6 ppm, 2.5 ppm, 1.8 ppm, 1.2 ppm.

### Compound I Form 5B

Compound I Form 5B is a crystalline form of an acetone solvate of the hydrochloride salt of Compound I. Form 5B is characterized by an XRPD comprising peaks (±0.2°) at 10.94, 17.62, 25.32, 26.46, and 27.30° 2θ, as determined on a diffractometer using Cu-Kα radiation. In some embodiments, the diffractogram comprises one, two, three, four, five, six, seven, eight, nine, ten, eleven, twelve, or thirteen more peaks selected from (±0.2°) 6.5, 9.4, 12.3, 12.9, 18.7, 19.3, 19.7, 21.2, 21.8, 33.6, 35.8, 37.0, and 39.1° 2θ. In some embodiments, the diffractogram comprises one, two, three, four, five, six, seven, eight, or nine additional peaks selected from (±0.2°) 14.9, 23.3, 23.7, 24.7, 27.7, 29.0, 29.5, 31.7, and 35.0° 2θ. Form 5B is also characterized by XRPD substantially as shown in FIG. 13B. In one embodiment, this disclosure provides Compound I Form 5B comprising two or more peaks (±0.2°) listed herein as determined on a diffractometer using Cu-Kα radiation.

In some embodiments, Form 5B is also characterized by TGA comprising a thermogram substantially as shown in FIG. 14B. As illustrated, Form 5B presents one weight loss step from room temperature to about 180° C., during which there is a weight loss of about 15%.

In some embodiments, Form 5B is further characterized by DSC curve substantially as shown in FIG. 15B. As illustrated. Form 5B presents two endothermic peaks and one exothermic peak. The first endothermic peak has an onset at about 102° C., a peak at about 113° C., and an endset at about 121° C. The second endothermic peak has an onset at about 233° C., a peak at about 249° C., and an endset at about 254° C. It is attributed to the melting. The exothermic peak has an onset at about 258° C., a peak at about 267° C., and an endset at about 283° C. It is attributed to compound decomposition.

In some embodiment, Form 5B is further characterized by ¹HNMR spectrum comprising peaks at 9.2 ppm, 8.2 ppm, 8.1 ppm, 8.0 ppm, 7.5 ppm, 2.6 ppm, 2.5 ppm, 1.2 ppm.

### Compound I Form 5C

Compound I Form 5C is a crystalline form of a methanol solvate of the hydrochloride salt of Compound I. Form 5C is characterized by an XRPD comprising peaks (±0.2°) at 14.6, 15.9, 19.3, 27.9, and 29.2°2θ, as determined on a diffractometer using Cu-Kα radiation. In some embodiments, the diffractogram comprises one, two, three, four, five, six, or seven more peaks selected from (±0.2°) 11.4, 12.7, 17.1, 22.2, 25.5, 25.9, and 27.2° 2θ. In some embodiments, the diffractogram comprises one, two, three, four, five, six, seven, eight, nine, ten, eleven, twelve, or thirteen additional peaks selected from) (±0.2°) 32.1, 33.5, 34.1, 36.7, 37.5, and 37.9°2θ. Form 5C is also characterized by XRPD substantially as shown in FIG. 13C. In one embodiment, this disclosure provides Compound I Form 5C comprising two or more peaks (±0.2°) listed herein as determined on a diffractometer using Cu-Kα radiation.

In some embodiments, Form 5C is also characterized by TGA comprising a thermogram substantially as shown in FIG. 14C. As illustrated, Form 5C presents two weight loss steps. The first step is from room temperature to about 120° C., during which there is a weight loss of about 1.3%. The second weight loss is from 120° C. to 236° C., during which there is a weight loss of about 4.6%.

In some embodiments, Form 5C is further characterized by DSC curve substantially as shown in FIG. 15C. As illustrated, Form 5C presents two endothermic peaks and one exothermic peak. The first endothermic peak has an onset at about 154° C., a peak at about 164° C., and an endset at about 172° C. The second endothermic peak has an onset at about 184° C., a peak at about 202° C., and an endset at about 213° C. Itis attributed to the melting. The exothermic peak has an onset at about 254° C., a peak at about 272° C., and an endset at about 287° C. It is attributed to compound decomposition.

### Compound I Form 5D

Compound I Form 5D is a crystalline form of anhydrous hydrochloride salt of Compound I. Form 5D is characterized by an XRPD comprising peaks (±0.2°) at 12.6, 14.4, 15.9, 22.0, 23.0, 27.0, 27.7, and 29.6° 2θ, as determined on a diffractometer using Cu-Kα radiation. In some embodiments, the diffractogram comprises one, two, three, four, five, six, seven, eight, nine, ten, eleven, twelve, or thirteen more peaks selected from (±0.2°) 9.6, 11.4, 16.7, 18.1, 19.1, 20.3, 24.0, 25.4, 28.6, 30.1, 31.7, 32.4, and 33.5° 2θ. Form 5D is also characterized by XRPD substantially as shown in FIG. 13D. In one embodiment, this disclosure provides Compound I Form 5D comprising two or more peaks (±0.2°) listed herein as determined on a diffractometer using Cu-Kα radiation.

In some embodiments, Form 5D is also characterized by TGA comprising a thermogram substantially as shown in FIG. 14D. As illustrated, Form 5D presents two weight loss steps. The first step is from room temperature to about 120° C., during which there is a weight loss of about 0.4%. The second weight loss is from 120° C. to 210° C., during which there is a weight loss of about 4%.

In some embodiments, Form 5D is further characterized by DSC curve substantially as shown in FIG. 15D. As illustrated, Form 5D presents one endothermic peaks and one exothermic peak. The endothermic peak has an onset at about 182° C., a peak at about 197° C., and an endset at about 210° C. Itis attributed to the melting. The exothermic peak has an onset at about 253° C., a peak at about 273° C., and an endset at about 289° C.′. It is attributed to compound decomposition.

In some embodiment, Form 5D is further characterized by 1HNMR spectrum comprising peaks at 9.1 ppm, 8.2 ppm, 8.0 ppm, 7.9 ppm, 7.4 ppm, 2.6 ppm, 1.2 ppm.

### Compound I Form 6

Compound I Form 6 is a crystalline form of the mesylate salt of Compound I. Form 6 is characterized by XRPD comprising peaks (±0.2°) at 15.9, 20.6, 24.2, 24.5, 25.8, 26.8, and 30.4°2θ, as determined on a diffractometer using Cu-Kα radiation. The diffractogram comprises one, two, three, four, five, six, seven, eight, nine, ten, or eleven additional peaks selected from (±0.2°) 9.1, 14.8, 15.5, 17.3, 19.8, 23.5, 26.1, 28.0, 28.4, 31.7, and 36.3° 2θ. Form 6 is also characterized by XRPD substantially as shown in FIG. 16. In one embodiment, this disclosure provides Compound I Form 6 comprising two or more peaks (±0.2°) listed herein as determined on a diffractometer using Cu-Kα radiation.

In some embodiments, Form 6 is also characterized by TGA comprising a thermogram substantially as shown in FIG. 17. As illustrated, Form 6 presents one weight loss step from room temperature to about 128° C., during which there is a weight loss of about 3.7%.

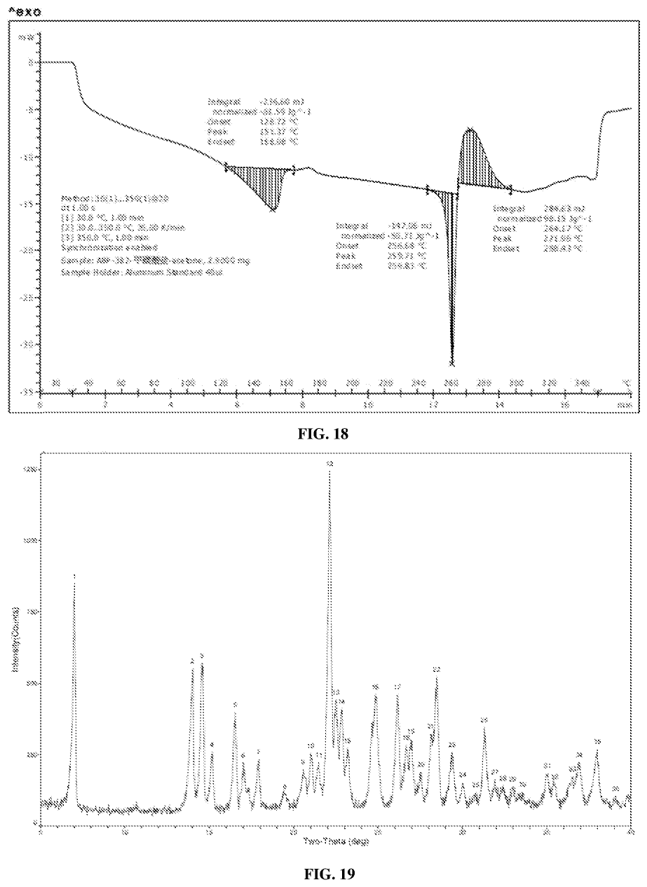

In some embodiments, Form 6 is further characterized by DSC curve substantially as shown in FIG. 18. As illustrated, Form 6 presents two endothermic peaks. The first endothermic peak has an onset at about 129° C.′, peaks at about 151° C., and ends at about 158° C. and is attributed to water escaping from the crystal. The second endothermic peak has an onset at about 257° C., peaks at about 260° C., and is attributed to the melting accompanied. The exothermic peak has an onset at about 264° C., and is attributed to compound decomposition. In some embodiments, Form 6 is a hydrate.

### Compound I Form 7

Compound I Form 7 is a crystalline form of the besylate salt of Compound I. Form 7 is characterized by an XRPD comprising peaks (±0.2°) at 7.0, 14.0, 14.6, 16.5, 22.1, 22.5, 22.8, 24.8, 26.1, and 28.5° 2θ, as determined on a diffractometer using Cu-Kα radiation. The diffractogram comprises one, two, three, four, five, six, seven, eight, nine, ten, eleven, twelve, thirteen, fourteen, fifteen, or sixteen additional peaks selected from) (+0.2° 15.2, 17.0, 17.9, 20.6, 21.0, 21.5, 23.2, 26.7, 26.9, 28.1, 29.4, 31.3, 35.1, 36.5, 36.9, and 38.0° 2θ. Form 7 is also characterized by XRPD substantially as shown in FIG. 19. In one embodiment, this disclosure provides Compound I Form 7 comprising two or more peaks (±0.2°) listed herein as determined on a diffractometer using Cu-Kα radiation.

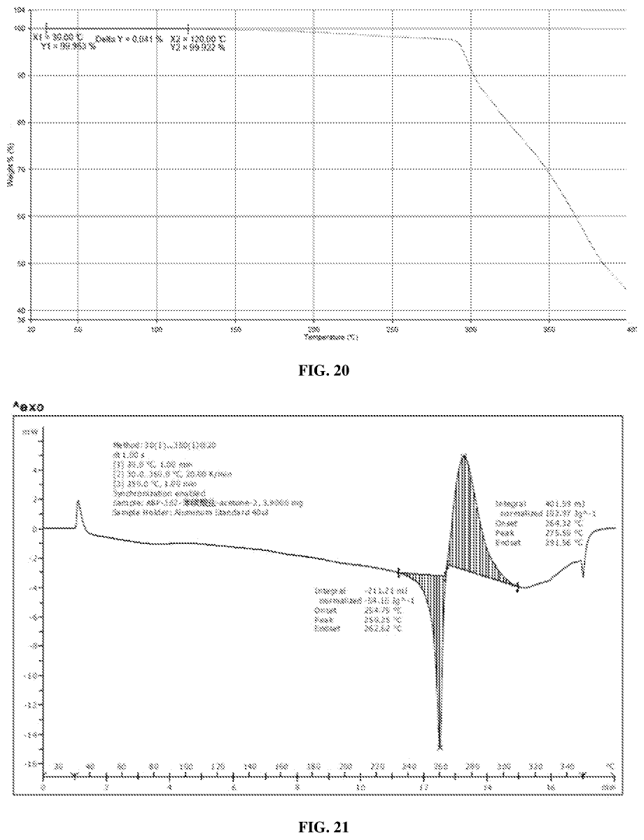

In some embodiments, Form 7 is also characterized by TGA comprising a thermogram substantially as shown in FIG. 20. As illustrated, Form 7 presents no significant weight loss until about 120° C.

In some embodiments, Form 7 is further characterized by DSC curve substantially as shown in FIG. 21. As illustrated, Form 7 presents one endothermic peak with an onset at about 255° C., peaks at about 259° C.′, and ends at about 263° C. It is attributed to the melting of the Form 7. The exothermic peak has an onset at about 264° C., and is attributed to compound decomposition.

In some embodiments, Form 7 is an anhydrous form of Compound I.

### Compound I Form 8

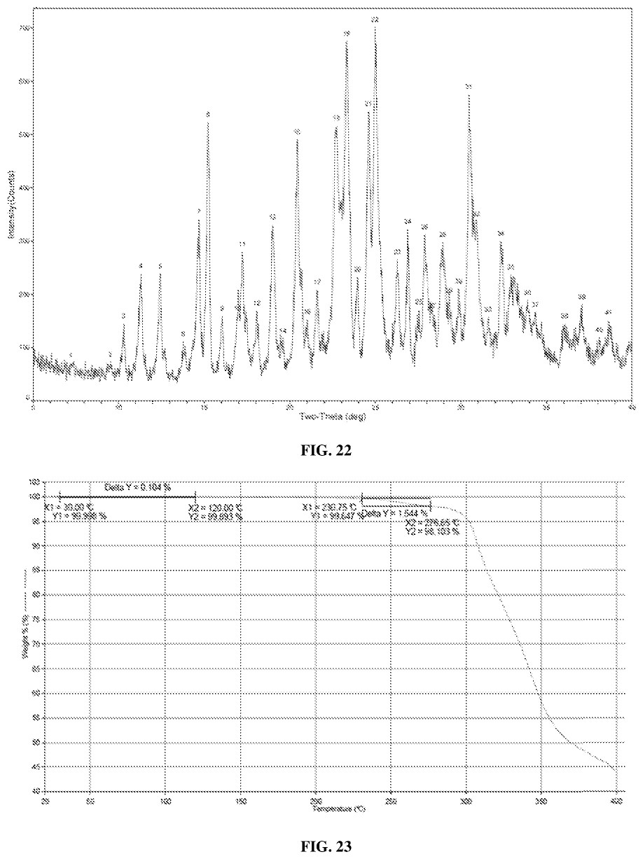

Compound I Form 8 is a crystalline form of the sulfate salt of Compound I. Form 8 is characterized by an XRPD comprising peaks (±0.2°) at 14.7, 15.2, 19.0, 20.4, 22.7, 23.3, 24.6, 25.0, 26.9, 30.5° 2θ, as determined on a diffractometer using Cu-Kα radiation. The diffractogram comprises one, two, three, four, five, or six additional peaks selected from (±0.2°) 16.9, 17.2, 27, 9, 28.9, and 30.9, 2θ. Form 8 is also characterized by XRPD substantially as shown in FIG. 22. In one embodiment, this disclosure provides Compound I Form 8 comprising two or more peaks (±0.2°) listed herein as determined on a diffractometer using Cu-Kα radiation.

In some embodiments, Form 8 is also characterized by TGA comprising a thermogram substantially as shown in FIG. 23. As illustrated, Form 8 presents no substantial weight loss until about 231° C.

In some embodiments, Form 8 is further characterized by DSC curve substantially as shown in FIG. 24. As illustrated, Form 8 presents one endothermic peak with an onset at about 238° C., peaks at about 253° C.′, and ends at about 247° C. It is attributed to the melting of Form 8.

In some embodiments, Form 8 is an anhydrous form of Compound I.

### Compound I Form 9

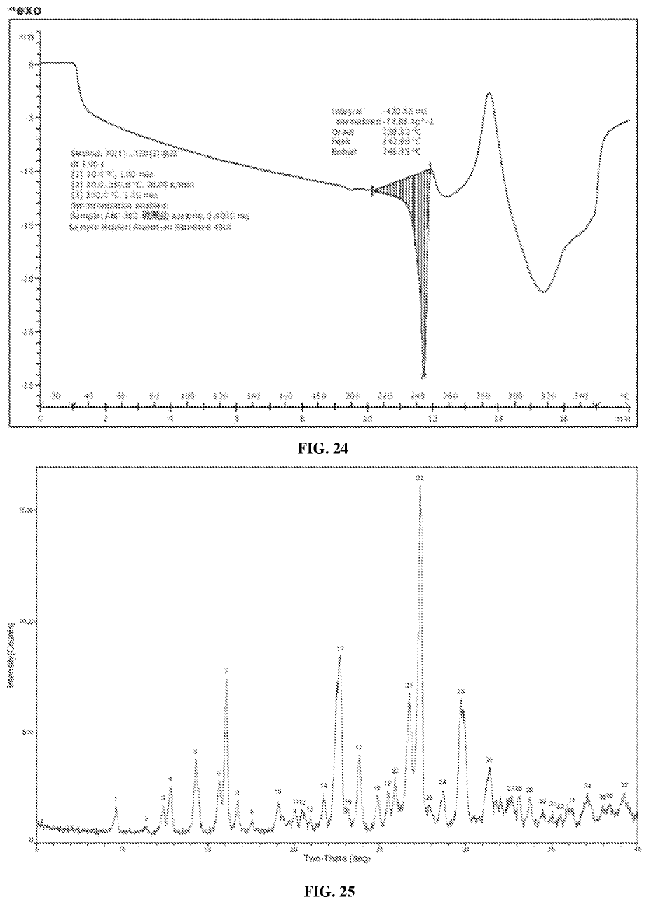

Compound I Form 9 is a crystalline form of the nitrate salt of Compound I. Form 9 is characterized by an XRPD comprising peaks (±0.2°) at 14.3, 16.1, 22.7, 23.8, 26.7, 27.4, and 29.7°2θ, as determined on a diffractometer using Cu-Kα radiation. The diffractogram comprises one, two, three, four, five, six, seven, eight, nine, ten, eleven, twelve, thirteen, fourteen, fifteen, sixteen, seventeen, or eighteen additional peaks selected from (±0.2°) 9.6, 12.4, 12.8, 15.6, 16.7, 19.1, 20.5, 21.7, 24.8, 25.5, 25.9, 28.7, 31.4, 32.6, 33.1, 33.8, 37.1, and 39.2° 2θ. Form 9 is also characterized by XRPD substantially as shown in FIG. 25. In one embodiment, this disclosure provides Compound I Form 9 comprising two or more peaks (±0.2°) listed herein as determined on a diffractometer using Cu-Kα radiation.

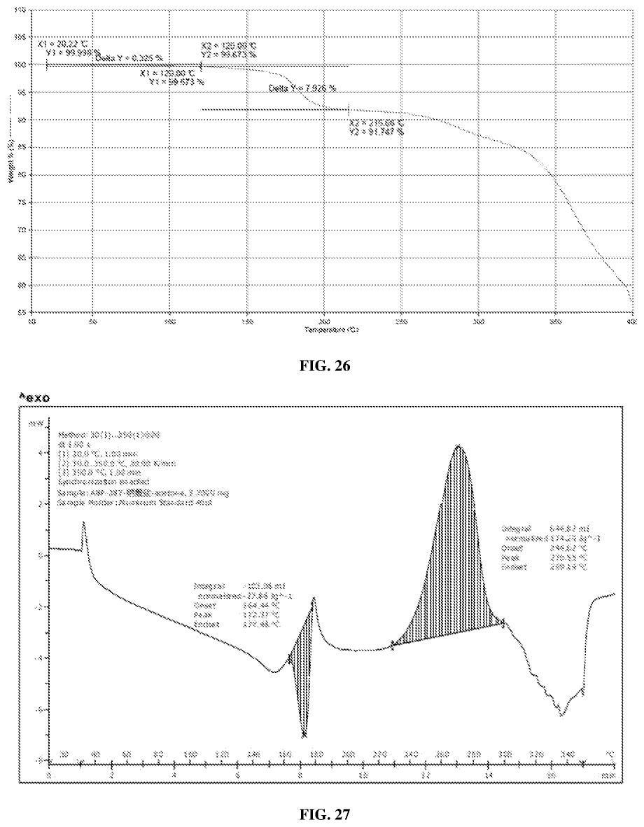

In some embodiments, Form 9 is also characterized by TGA comprising a thermogram substantially as shown in FIG. 26. As illustrated, Form 9 presents a first weight loss step from room temperature to about 120° C., during which there is a weight loss of about 0.3%. Form 9 further presents a second weight loss step from about 120° C. to about 216° C., during which there is a weight loss of about 8%.

In some embodiments, Form 9 is further characterized by DSC curve substantially as shown in FIG. 27. As illustrated, Form 9 presents an endothermic peak with an onset at about 164° C., peaks at about 172° C., and ends at about 178° C. It is attributed to the melting of Form 9. Form 9 further presents an exothermic peak with an onset at about 245° C.′, peaking at about 271° C., and ending at about 289° C. It is attributed to compound decomposition.

In some embodiments, Form 9 is an anhydrous form of Compound I.

### Compound I Form 10

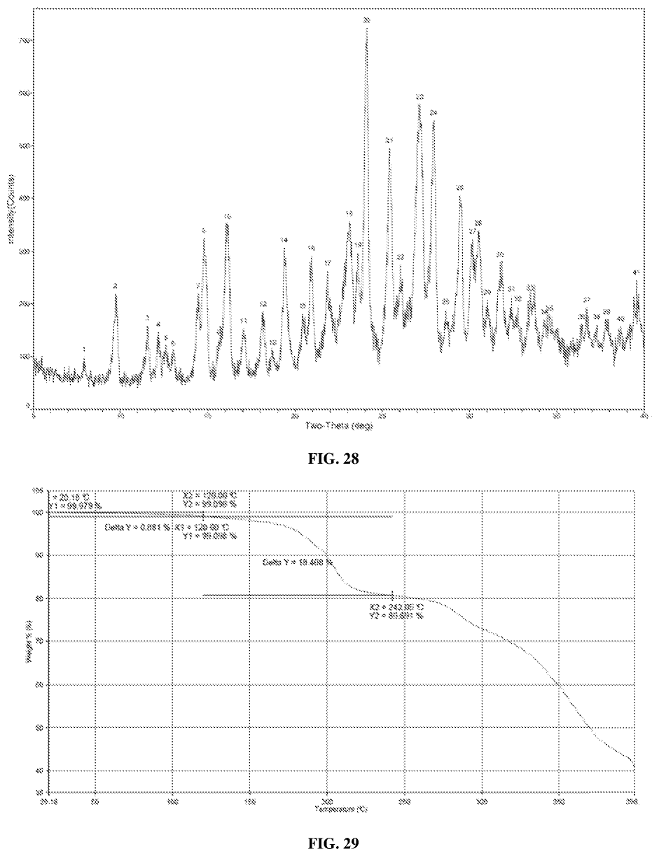

Compound I Form 10 is a crystalline form of the maleate salt of Compound I. Form 10 is characterized by an XRPD comprising peaks (±0.2°) at 9.7, 14.8, 16.1, 19.4, 20.9, 23.1, 24.1, 25.4, 27.1, 28.0, 29.4, 30.2, and 30.5° 2θ, as determined on a diffractometer using Cu-Kα radiation. The diffractogram comprises one, two, three, four, five, six, seven, eight, nine, ten, eleven, or twelve additional peaks selected from (±0.2°) 11.6, 12.2, 14.5, 17.0, 18.2, 20.4, 21.9, 23.6, 26.0, 31.7, 32.4, 33.5, and 39.6° 2θ. Form 10 is also characterized by XRPD substantially as shown in FIG. 28. In one embodiment, this disclosure provides Compound I Form 10 comprising two or more peaks (±0.2°) listed herein as determined on a diffractometer using Cu-Kα radiation.

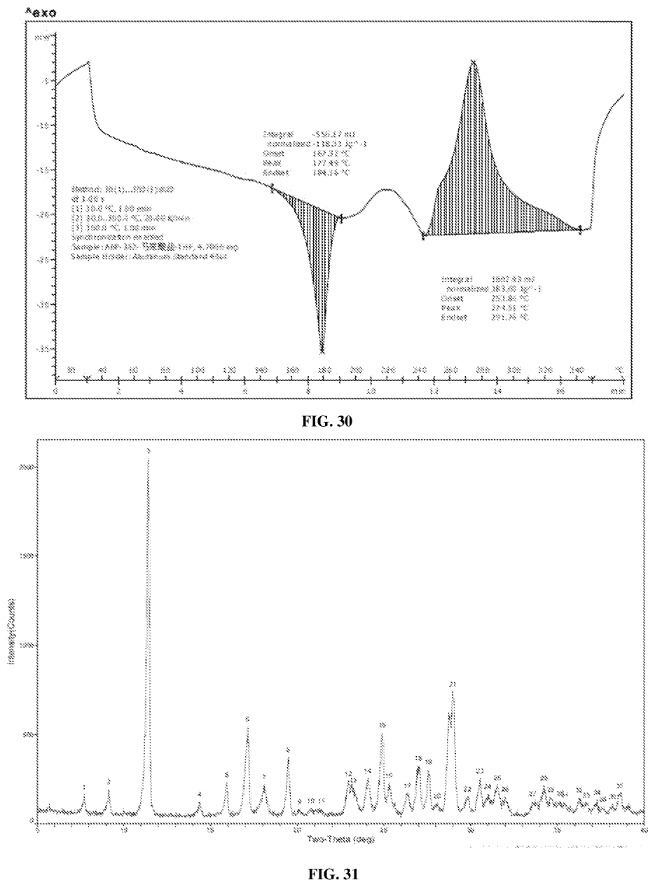

In some embodiments, Form 10 is also characterized by TGA comprising a thermogram substantially as shown in FIG. 29. As illustrated, Form 10 presents a first weight loss step from room temperature to about 120° C. during which there is a weight loss of about 0.2%. Form 10 further presents a second weight loss step from about 120° C. to about 242° C., during which there is a weight loss of about 18.4%.

In some embodiments, Form 10 is further characterized by DSC curve substantially as shown in FIG. 30. As illustrated, Form 10 presents an endothermic peaks with an onset at about 167° C., peaking at about 178° C., and ending at about 184° C. It is attributed to the melting and decomposition of Form 10.

In some embodiments, Form 10 is an anhydrous form of Compound I.

### Compound I Form 11

Compound I Form 11 is a crystalline form of the potassium salt of Compound I. Form 11 is characterized by an XRPD comprising peaks (±0.2°) at 11.4, 17.1, 19.5, 24.9, 27.0, 27.6, and 29.0° 2θ, as determined on a diffractometer using Cu-Ko radiation. The diffractogram comprises one, two, three. four, five, six, seven, eight, nine, ten, eleven, twelve, thirteen, fourteen, fifteen, sixteen, seventeen, eighteen, or nineteen additional peaks selected from (±0.2°) 7.7, 9.1, 14.4, 15.9, 18.1, 22.9, 23.2, 24.1, 25.3, 26.4, 29.8, 30.5, 31.0, 31.6, 32.0, 33.6, 34.2, 36.3, and 38.6° 2θ. Form 11 is also characterized by XRPD substantially as shown in FIG. 31. In one embodiment, this disclosure provides Compound I Form 11 comprising two or more peaks (±0.2°) listed herein as determined on a diffractometer using Cu-Kα radiation.

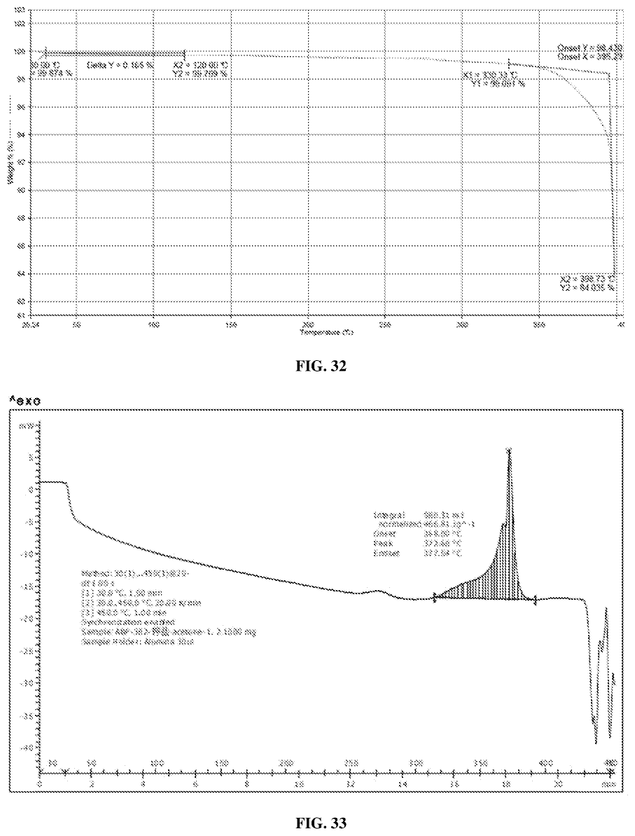

In some embodiments, Form 11 is also characterized by TGA comprising a thermogram substantially as shown in FIG. 32. As illustrated, Form 11 presents no substantial weight loss step. For example, the weight loss from room temperature to about 120° C. is only about 0.2%.

In some embodiments, Form 11 is further characterized by DSC curve substantially as shown in FIG. 33. As illustrated, Form 11 presents an exothermic peak with an onset of about 368° C., peaking at about 373° C.′ and ending at about 378° C. It is attributed to compound decomposition.

In some embodiments, Form 11 is an anhydrous form of Compound I.

### Compound I Form 12

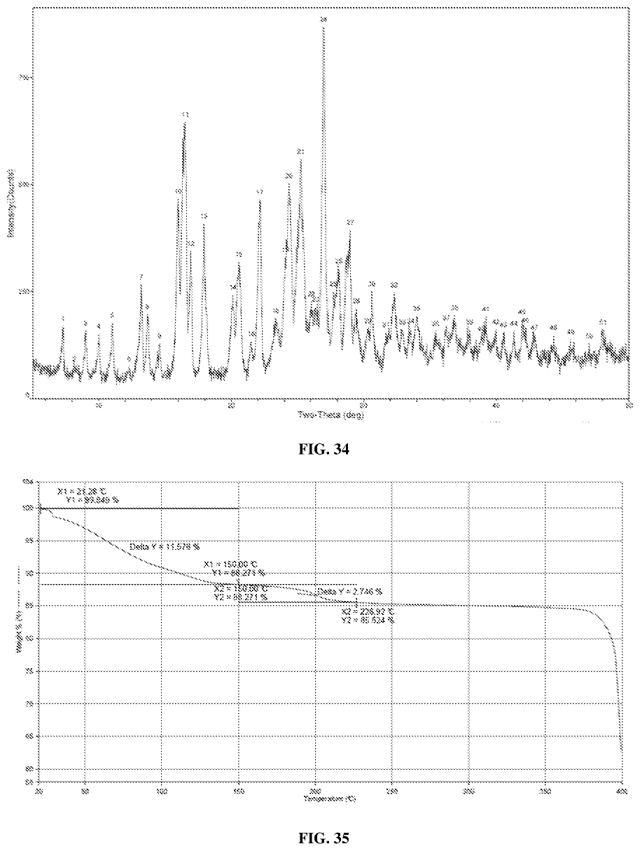

Compound I Form 12 is a crystalline form of the sodium salt of Compound I. Form 12 is characterized by an XRPD comprising peaks (±0.2°) at 13.2, 16.0, 16.5, 16.9, 17.9, 20.6, 22.1, 24.4, 25.3, 27.0, and 29.0° 2θ, as determined on a diffractometer using Cu-Kα radiation. The diffractogram comprises one, two, three, four, five, six, seven, eight, nine, or ten additional peaks selected from (±0.2°) 7.3, 9.0, 10.0, 11.0, 13.7, 20.1, 24.1, 27.7, 28.1, 30.6, and 32.3°2θ. Form 12 is also characterized by XRPD substantially as shown in FIG. 34. In one embodiment, this disclosure provides Compound I Form 12 comprising two or more peaks (±0.2°) listed herein as determined on a diffractometer using Cu-Kα radiation.

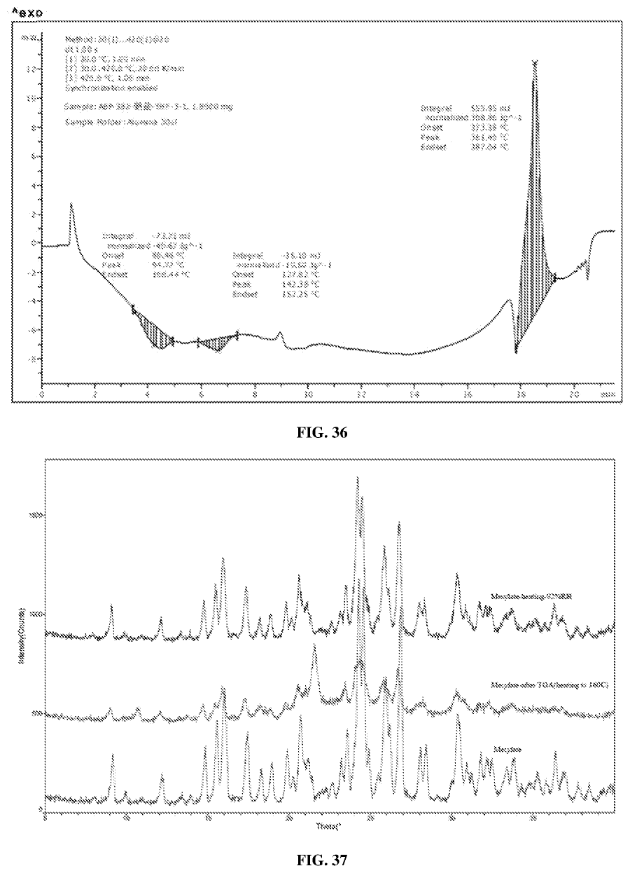

In some embodiments, Form 12 is also characterized by TGA comprising a thermogram substantially as shown in FIG. 35A or 35B. In some embodiments, Form 12 is further characterized by DSC curve substantially as shown in FIG. 36A or 36B. FIG. 35A and FIG. 36A are received from the analysis of Form 12 prepared from THE as the solvent; while FIG. 35B and FIG. 36B are received the analysis of Form 12 prepared from acetone.

As illustrated in FIG. 35A, Form 12 (from THF) presents one weight loss step from room temperature to about 150° C., during which there is a weight loss of about 11.6%. It further presents another weight loss step from about 150° C. to about 227° C.′. Moreover, as illustrated in FIG. 36A, Form 12 (from THF) presents two endothermic peaks. The first endothermic peak has an onset at about 80° C., peaks at about 95° C., and ends at about 108° C.′. The second endothermic peak has an onset at about 128° C., peaks at about 142 C. and ends at about 152° C. Both endothermic peaks are attributed to solvent loss from the crystal. Form 12 further presents an exothermic peak having an onset at about 373° C., peaking at about 381° C., and ending at about 387° C. It is attributed to compound decomposition.

As illustrated in FIG. 35B, Form 12 (from acetone) presents one weight loss step from room temperature to about 150° C., during which there is a weight loss of about 8.8%. Moreover, as illustrated in FIG. 36B, Form 12 (from acetone) presents two endothermic peaks. The first endothermic peak has an onset at about 87° C., peaks at about 107° C., and ends at about 114° C.′. The second endothermic peak has an onset at about 116° C., peaks at about 137° C., and ends at about 165° C. Both endothermic peaks are attributed to solvent loss from the crystal.

In some embodiments, Form 12 includes water in its crystalline structure.

### Compositions

In one embodiment, this disclosure provides a composition comprising two or more compounds selected from the group consisting of Compound I Form 1, Compound I Form 2, Compound I Form 3, Compound I Form 4, Compound I Form 5A, Compound I Form 5B, Compound I Form 5C, Compound I Form 5D, Compound I Form 6, Compound I Form 7, Compound I Form 8, Compound I Form 9, Compound I Form 10, Compound I Form 11, and Compound I Form 12 as described herein.

In another embodiment, the composition comprises Compound I Form 1, Compound I Form 2. Compound I Form 3, Compound I Form 4, Compound I Form 5A, Compound I Form 5B, Compound I Form 5C, Compound I Form 5D, Compound I Form 6, Compound I Form 7, Compound I Form 8, Compound I Form 9, Compound I Form 10, Compound I Form 11, or Compound I Form 12. In another embodiment, the composition comprises Compound I wherein at least about 10%, 15%, 20%, 25%, 30%, 35%, 40%, 45%, 50%, 55%, 60%, 65%, 70%, 75%, 80%, 85% w/w of Compound I is Form 1. In another embodiment, the composition comprises Compound I wherein at least 90%, 95% w/w of Compound I is Form 1. In another embodiment, the composition comprises Compound I wherein at least about 10%, 15%, 20%, 25%, 30%, 35%, 40%, 45%, 50%, 55%, 60%, 65%, 70%, 75%, 80%, 85% w/w of Compound I is Form 2. In another embodiment, the composition comprises Compound I wherein at least 90%, 95% w/w of Compound I is Form 2. In another embodiment, the composition comprises Compound I wherein at least about 10%, 15%, 20%, 25%, 30%, 35%, 40%, 45%, 50%, 55%, 60%, 65%, 70%, 75%, 80%, 85% w/w of Compound I is Form 3. In another embodiment, the composition comprises Compound I wherein at least 90%, 95% w/w of Compound I is Form 3. In another embodiment, the composition comprises Compound I wherein at least about 10%, 15%, 20%, 25%, 30%, 35%, 40%, 45%, 50%, 55%, 60%, 65%, 70%, 75%, 80%, 85% w/w of Compound I is Form 4. In another embodiment, the composition comprises Compound I wherein at least 90%, 95% w/w of Compound I is Form 4. In another embodiment, the composition comprises Compound I wherein at least about 10%, 15%, 20%, 25%, 30%, 35%, 40%, 45%, 50%, 55%, 60%, 65%, 70%, 75%, 80%, 85% w/w of Compound I is Form 5A. In another embodiment, the composition comprises Compound I wherein at least 90%, 95% w/w of Compound I is Form 5A. In another embodiment, the composition comprises Compound I wherein at least about 10%, 15%, 20%, 25%, 30%, 35%, 40%, 45%, 50%, 55%, 60%, 65%, 70%, 75%, 80%, 85% w/w of Compound I is Form 5B. In another embodiment, the composition comprises Compound I wherein at least 90%, 95% w/w of Compound I is Form 5B. In another embodiment, the composition comprises Compound I wherein at least about 10%, 15%, 20%, 25%, 30%, 35%, 40%, 45%, 50%, 55%, 60%, 65%, 70%, 75%, 80%, 85% w/w of Compound I is Form 5C. In another embodiment, the composition comprises Compound I wherein at least 90%, 95% w/w of Compound I is Form 5C. In another embodiment, the composition comprises Compound I wherein at least about 10%, 15%, 20%, 25%, 30%, 35%, 40%, 45%, 50%, 55%, 60%, 65%, 70%, 75%, 80%, 85% w/w of Compound I is Form 5D. In another embodiment, the composition comprises Compound I wherein at least 90%, 95% w/w of Compound I is Form 5D. In another embodiment, the composition comprises Compound I wherein at least about 10%, 15%, 20%, 25%, 30%, 35%, 40%, 45%, 50%, 55%, 60%, 65%, 70%, 75%, 80%, 85% w/w of Compound I is Form 6. In another embodiment, the composition comprises Compound I wherein at least 90%, 95% w/w of Compound I is Form 6. In another embodiment, the composition comprises Compound I wherein at least about 10%, 15%, 20%, 25%, 30%, 35%, 40%, 45%, 50%, 55%, 60%, 65%, 70%, 75%, 80%, 85% w/w of Compound I is Form 7. In another embodiment, the composition comprises Compound I wherein at least 90%, 95% w/w of Compound I is Form 7. In another embodiment, the composition comprises Compound I wherein at least about 10%, 15%, 20%, 25%, 30%, 35%, 40%, 45%, 50%, 55%, 60%, 65%, 70%, 75%, 80%, 85% w/w of Compound I is Form 8. In another embodiment, the composition comprises Compound I wherein at least 90%, 95% w/w of Compound I is Form 8. In another embodiment, the composition comprises Compound I wherein at least about 10%, 15%, 20%, 25%, 30%, 35%, 40%, 45%, 50%, 55%, 60%, 65%, 70%, 75%, 80%, 85% w/w of Compound I is Form 9. In another embodiment, the composition comprises Compound I wherein at least 90%, 95% w/w of Compound I is Form 9. In another embodiment, the composition comprises Compound I wherein at least about 10%, 15%, 20%, 25%, 30%, 35%, 40%, 45%, 50%, 55%, 60%, 65%, 70%, 75%, 80%, 85% w/w of Compound I is Form 10. In another embodiment, the composition comprises Compound I wherein at least 90%, 95% w/w of Compound I is Form 10. In another embodiment, the composition comprises Compound I wherein at least about 10%, 15%, 20%, 25%, 30%, 35%, 40%, 45%, 50%, 55%, 60%, 65%, 70%, 75%, 80%, 85% w/w of Compound I is Form 11. In another embodiment, the composition comprises Compound I wherein at least 90%, 95% w/w of Compound I is Form 11. In another embodiment, the composition comprises Compound I wherein at least about 10%, 15%, 20%, 25%, 30%, 35%, 40%, 45%, 50%, 55%, 60%, 65%, 70%, 75%, 80%, 85% w/w of Compound I is Form 12. In another embodiment, the composition comprises Compound I wherein at least 90%, 95% w/w of Compound I is Form 12.

In another embodiment, provided is a composition comprising Compound I Form land Compound I Form 2. In another embodiment, the composition comprises at least 50% w/w of Compound I Form 2.

In another embodiment, provided is a composition comprising Compound I, wherein at least 85%, or at least 85%, or at least 90%, or at least 95%, or at least 97%, or at least 98%, or at least 99%, or at least 99.5%, of the Compound I in the composition is present as Compound I Form 2. In another embodiment, the composition comprising Compound I Form 2 is not substantially changed after at least about 6 months, or 12 months, or 24 months, or 36 months, or 48 months. In another embodiment, the composition comprising Compound I Form 2 is not substantially changed after about 6 months at 40° C.±2° C., optionally at 75% RH±5% RH. In another embodiment, the composition comprising Compound I Form 2 is not substantially changed after for 6 months, or 12 months, or 24 months, or 36 months, or 48 months. In another embodiment, the composition comprising Compound I Form 2 is not substantially changed after for 6 months, or 12 months, or 24 months, or 36 months, or 48 months at 25° C.±2° C./60% RH±5% RH.

### Formulations and Administration

In another aspect, the present disclosure provides pharmaceutical compositions comprising/including a pharmaceutically acceptable carrier or excipient and a Compound I Form as described herein. In an exemplary embodiment, the present disclosure provides a pharmaceutical composition (or interchangeably “formulation”) comprising Compound I Form 1, Compound I Form 2, Compound I Form 3, Compound I Form 4, Compound I Form 5A, Compound I Form 5B, Compound I Form 5C, Compound I Form 5D, Compound I Form 6, Compound I Form 7, Compound I Form 8, Compound I Form 9, Compound I Form 10, Compound I Form 11, or Compound I Form 12 as described herein.

The forms will typically be used in therapy for human subjects. However, they may also be used to treat similar or identical indications in other animal subjects. The solid, crystalline, or polymorphs of Compound I described herein can be administered by different routes, including injection (i.e. parenteral, including intravenous, intraperitoneal, subcutaneous, and intramuscular), oral, transdermal, transmucosal, rectal, or inhalant. Such dosage forms should allow the compound to reach target cells. Other factors are well known in the art, and include considerations such as toxicity and dosage forms that retard the compound or composition from exerting its effects. Techniques and formulations generally may be found in Remington: The Science and Practice of Pharmacy, 21st edition, Lippincott, Williams and Wilkins, Philadelphia, PA. 2005 (hereby incorporated by reference herein).

In some embodiments, the compositions comprise one or more of Compound I Form 1, Compound I Form 2, Compound I Form 3, Compound I Form 4, Compound I Form 5A, Compound I Form 5B, Compound I Form 5C, Compound I Form 5D, Compound I Form 6, Compound I Form 7, Compound I Form 8, Compound I Form 9, Compound I Form 10, Compound I Form 11, and Compound I Form 12 with a particular particle size. In some embodiments, particle size critically affects bioavailability. In some embodiments, the compositions comprises the solid forms described here with particle size of about 1 nm to about 500 μm. In some embodiments, the compositions comprises the solid forms described here with particle size of about 1 nm to about 100 μm. In some embodiments, the compositions comprises the solid forms described here with particle size of about 1 nm to about 75 μm. In some embodiments, the compositions comprises the solid forms described here with particle size of about 1 nm to about 50 μm. In some embodiments, the compositions comprises the solid forms described here with particle size of about 1 nm to about 20 μm. In some embodiments, the compositions comprises the solid forms described here with particle size of about 1 nm to about 10 μm. In some embodiments, the compositions comprises the solid forms described here with particle size of about 1 nm to about 5 μm. In some embodiments, the compositions comprises the solid forms described here with particle size of about 1 nm to about 1 μm. In some embodiments, the compositions comprises the solid forms described here with particle size of about 1 nm to about 100 nm. In some embodiments, the compositions comprises the solid forms described here with particle size of about 1 nm to about 50 nm. In some embodiments, the compositions comprises the solid forms described here with particle size of about 1 nm to about 20 nm. In some embodiments, the compositions comprises the solid forms described here with particle size of about 1 nm to about 10 nm. In some embodiments, the desired particle size is achieved by implementing a homogenization step, such as a grinding, sonication, or other similar operation. In some embodiments, the length and intensity of the sonification is controlled to fine tune the desired particle size.

In some embodiments, the compositions will comprise pharmaceutically acceptable carriers or excipients, such as fillers, binders, disintegrants, glidants, lubricants, complexing agents, solubilizers, and surfactants, which may be chosen to facilitate administration of the compound by a particular route. Examples of carriers include calcium carbonate, calcium phosphate, various sugars such as lactose, glucose, or sucrose, types of starch, cellulose derivatives, gelatin, lipids, liposomes, nanoparticles, and the like. Carriers also include physiologically compatible liquids as solvents or for suspensions, including. for example, sterile solutions of water for injection (WFI), saline solution, dextrose solution, Hank's solution, Ringer's solution, vegetable oils, mineral oils, animal oils, polyethylene glycols, liquid paraffin, and the like. Excipients may also include, for example, colloidal silicon dioxide, silica gel, talc, magnesium silicate, calcium silicate, sodium aluminosilicate, magnesium trisilicate, powdered cellulose, macrocrystalline cellulose, carboxymethyl cellulose, cross-linked sodium carboxymethylcellulose, sodium benzoate, calcium carbonate, magnesium carbonate, stearic acid, aluminum stearate, calcium stearate, magnesium stearate, zinc stearate, sodium stearyl fumarate, syloid, stearowet C, magnesium oxide, starch, sodium starch glycolate, glyceryl monostearate, glyceryl dibehenate, glyceryl palmitostearate, hydrogenated vegetable oil, hydrogenated cotton seed oil, castor seed oil mineral oil, polyethylene glycol (e.g. PEG 4000-8000), polyoxyethylene glycol, poloxamers, povidone, crospovidone, croscarmellose sodium, alginic acid, casein, methacrylic acid divinylbenzene copolymer, sodium docusate, cyclodextrins (e.g. 2-hydroxypropyl-delta-cyclodextrin), polysorbates (e.g. polysorbate 80), cetrimide, TPGS (d-alpha-tocopherol polyethylene glycol 1000 succinate), magnesium lauryl sulfate, sodium lauryl sulfate, polyethylene glycol ethers, di-fatty acid ester of polyethylene glycols, or a polyoxyalkylene sorbitan fatty acid ester (e.g., polyoxyethylene sorbitan ester Tween®)), polyoxyethylene sorbitan fatty acid esters, sorbitan fatty acid ester, e.g. a sorbitan fatty acid ester from a fatty acid such as oleic, stearic or palmitic acid, mannitol, xylitol, sorbitol, maltose, lactose, lactose monohydrate or lactose spray dried, sucrose, fructose, calcium phosphate, dibasic calcium phosphate, tribasic calcium phosphate, calcium sulfate, dextrates, dextran, dextrin, dextrose, cellulose acetate, maltodextrin, simethicone, polydextrosem, chitosan, gelatin, HPMC (hydroxypropyl methyl celluloses), HPC (hydroxypropyl cellulose), hydroxyethyl cellulose, and the like.

Pharmaceutical compositions or formulations may be presented in unit dose forms containing a predetermined amount of active ingredient per unit dose. Such a unit may contain, for example, 0.5 mg to 1 g, preferably 1 mg to 700 mg, more preferably 5 mg to 100 mg of a solid, crystalline, or polymorph of Compound I of the disclosure, depending on the condition being treated, the route of administration, and the age, weight and condition of the patient. Preferred unit dosage formulations are those containing a daily dose, weekly dose, monthly dose, a sub-dose or an appropriate fraction thereof, of an active ingredient. Furthermore, such pharmaceutical compositions or formulations may be prepared by any of the methods well known in the pharmacy art.

Pharmaceutical compositions or formulations may be adapted for administration by any appropriate route, for example by the oral (including capsules, tablets, liquid-filled capsules, disintegrating tablets, immediate, delayed and controlled release tablets, oral strips, solutions, syrups, buccal and sublingual), rectal, nasal, inhalation, topical (including transdermal), or parenteral (including subcutaneous, intramuscular, intravenous or intradermal) route. Such formulations may be prepared by any method known in the art of pharmacy, for example by bringing into association the active ingredient with the carrier(s), excipient(s) or diluent. Generally, the carrier, excipient or diluent employed in the pharmaceutical formulation is “non-toxic,” meaning that it/they is/are deemed safe for consumption in the amount delivered in the pharmaceutical composition, and “inert” meaning that it/they does/do not appreciably react with or result in an undesired effect on the therapeutic activity of the active ingredient.

In some embodiments, oral administration may be used. Pharmaceutical preparations for oral use can be formulated into conventional oral dosage forms such as discrete units capsules, tablets, and liquid preparations such as syrups, elixirs, and concentrated drops. Compounds described herein may be combined with solid excipients, optionally grinding a resulting mixture, and processing the mixture of granules, after adding suitable auxiliaries, if desired, to obtain, for example, tablets, coated tablets, hard capsules, soft capsules, solutions (e.g. aqueous, alcoholic, or oily solutions) and the like. Suitable excipients are, in particular, fillers such as sugars, including lactose, glucose, sucrose, mannitol, or sorbitol; cellulose preparations, for example, corn starch, wheat starch, rice starch, potato starch, gelatin, gum tragacanth, methyl cellulose, hydroxypropylmethyl-cellulose, sodium carboxymethylcellulose (CMC), and/or polyvinylpyrrolidone (PVP: povidone); oily excipients, including vegetable and animal oils, such as sunflower oil, olive oil, or cod liver oil. The oral dosage formulations may also contain disintegrating agents, such as the cross-linked polyvinylpyrrolidone, agar, or alginic acid, or a salt thereof such as sodium alginate; a lubricant, such as talc or magnesium stearate; a plasticizer, such as glycerol or sorbitol; a sweetening such as sucrose, fructose, lactose, or aspartame; a natural or artificial flavoring agent, such as peppermint, oil of wintergreen, or cherry flavoring; or dye-stuffs or pigments, which may be used for identification or characterization of different doses or combinations, such as unit dosages. Also provided are dragee cores with suitable coatings. For this purpose, concentrated sugar solutions may be used, which may optionally contain, for example, gum arabic, talc, poly-vinylpyrrolidone, carbopol gel, polyethylene glycol, and/or titanium dioxide, lacquer solutions, and suitable organic solvents or solvent mixtures. Oral fluids such as solutions, syrups and elixirs can be prepared in dosage unit form so that a given quantity contains a predetermined amount of the solid, crystalline, or polymorph of Compound I.

Pharmaceutical preparations that can be used orally include push-fit capsules made of gelatin (“gelcaps”), as well as soft, sealed capsules made of gelatin, and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols.

The amounts of various compounds to be administered can be determined by standard procedures taking into account factors such as the compound activity (in vitro, e.g. the compound IC₅₀ vs. target, or in vivo activity in animal efficacy models), pharmacokinetic results in animal models (e.g. biological half-life or bioavailability), the age, size, and weight of the subject, and the disorder associated with the subject. The importance of these and other factors are well known to those of ordinary skill in the art. Generally, a dose may be in the range of about 0.01 to 50 mg/kg, also about 0.1 to 20 mg/kg of the subject being treated. Multiple doses may be used.

The solid, crystalline, or polymorph of Compound I as described herein may also be used in combination with other therapies for treating the same disease. Such combination use includes administration of the compounds and one or more other therapeutics at different times, or co-administration of the compound and one or more other therapies. In some embodiments, dosage may be modified for one or more forms of the Compound I or other therapeutics used in combination, e.g., reduction in the amount dosed relative to a compound or therapy used alone, by methods well known to those of ordinary skill in the art.

It is understood that use in combination includes use with other therapies, drugs, medical procedures etc., where the other therapy or procedure may be administered at different times (e.g. within a short time, such as within hours (e.g. 1, 2, 3, 4-24 hours), or within a longer time (e.g. 1-2 days, 2-4 days, 4-7 days, 1-4 weeks)) than a compound described herein, or at the same time as a compound described herein. Use in combination also includes use with a therapy or medical procedure that is administered once or infrequently, such as surgery, along with a compound described herein administered within a short time or longer time before or after the other therapy or procedure. In some embodiments, the present disclosure provides for delivery of a Compound I form as described herein and one or more other drug therapeutics delivered by a different route of administration or by the same route of administration. The use in combination for any route of administration includes delivery of a compound described herein and one or more other drug therapeutics delivered by the same route of administration together in any formulation, including formulations where the two compounds are chemically linked in such a way that they maintain their therapeutic activity when administered. In one aspect, the other drug therapy may be co-administered with a compound described herein. Use in combination by co-administration includes administration of co-formulations or formulations of chemically joined compounds, or administration of two or more compounds in separate formulations within a short time of each other (e.g. within an hour, 2 hours, 3 hours, up to 24 hours), administered by the same or different routes. Co-administration of separate formulations includes co-administration by delivery via one device, for example the same syringe, etc., or administration from separate devices within a short time of each other. Co-formulations of a compound described herein and one or more additional drug therapies delivered by the same route includes preparation of the materials together such that they can be administered by one device, including the separate compounds combined in one formulation, or compounds that are modified such that they are chemically joined, yet still maintain their biological activity. Such chemically joined compounds may have a linkage that is substantially maintained in vivo, or the linkage may break down in vivo, separating the two active components.

### URAT1Targets and Indications

The solid forms of Compound I, such as Compound I Form 1, Compound I Form 2, Compound I Form 3, Compound I Form 4. Compound I Form 5A, Compound I Form 5B, Compound I Form 5C. Compound I Form 5D, Compound I Form 6, Compound I Form 7, Compound I Form 8, Compound I Form 9, Compound I Form 10, Compound I Form 11, and Compound I Form 12, alone or in combination with each other, may be used to treat or prevent various diseases, such as gout and hyperuricemia. Gout is a metabolic disease caused by chronically elevated serum uric acid (sUA) levels (hyperuricemia) due to the disorder of purine metabolism and/or from insufficient renal elimination of uric acid. Deposition of the needle-like crystals of urate in the joints leads to painful inflammatory arthritis. Hyperuricemia, defined as sUA concentration higher or equal to 6.8 mg/dL, may result in the precipitation of urate as mono-sodium salt in the synovial fluid of the human soft tissue, the cartilage of the peripheral joint, the auricle of the ear, and the olecranon bursa of the elbow. When such symptoms are presents, it can be diagnosed as gout. (Terkeltaub R A. Crystal Deposition Diseases. In: Goldman L, Aus-iello D, eds. The Cecil Textbook of Medicine, 23rd ed. Philadelphia, Pa.: Saunders Elsevier Co; 2008:2069-2075; Richette P, Bardin T. Gout. Lancet. 2010, 375 (9711): 318-328). Gout is the common type of inflammatory arthritis and has an incidence of approximately 1%-2%. The incidence in the developed countries is relatively high, as a survey of 2007-2008 reported there were about 8.3 million of gout patients in the US. In China, the incidence of gout has dramatically increased in the past decade. It is reported that the number of gout patients in China has exceeded 50 million, and the proportion of men with gout is much higher than that of women.

Uric acid excretion plays a very important role in the treatment of hyperuricemia and gout. Human urate anion transporter 1 (human URAT1 or hURAT1) is located in the proximal tubular epithelial cell membrane, and it belongs a super family member of an organic anion transporter (OAT), which is encoded by SLC22A12 gene. Its cDNA has several mutations that cause uric acid metabolism abnormally. A Meta analysis showed that this gene has 0.13% variables contributed to serum uric acid level. (So A, Thorens B. Uric acid transport and disease. Journal of Clinical Investigation., 2010, 120 (6): 1791-1799). The URAT1 controls more than 90% of the uric acid re-absorption after glomerular filtration. Therefore, selective inhibition of URAT1 can decrease the re-absorption of uric acid and promote the excretion of uric acid in the kidneys to reduce uric acid levels in the body. (Michael F W, Jutabha P, Quada B. Developing potent human uric acid transporter 1 (hURAT1) inhibitors. Journal of Medicinal Chemistry. 2011, 54:2701-2713). Due to high toxicity, low efficacy, or other side effects associated with presently known treatments of gout and hyperuricemia, it is critical to develop new drugs that are highly effective and have low toxicity.

Compound I is aURAT1 inhibitor. The test results both in vitro and in vivo showed that Compound I can significantly improve the inhibitory effect on URAT1, as well as significantly increase uric acid excretion in mice and reduce the toxicity to normal liver cells in comparison with other treatments. The oral maximum tolerated dose of acute toxicity test in rats showed that the toxicity of the compound provided by the invention was much lower than other treatments. The studies have shown that the compound provided by the invention is highly effective in uric acid excretion and has low toxicity.

To effectively use Compound I as a therapeutic agent, it would be desirable to have a solid form that can be readily manufactured and that has acceptable chemical and physical stability. For example, it would be highly desirable to have a solid form that is thermally stable, for example at temperatures exceeding about 240° C., and is not hygroscopic nor deliquescent, thereby facilitating processing and storage of the material. Crystalline solids are sometimes preferred over amorphous forms, for enhancing purity and stability of the manufactured product. Accordingly, a need exists for a stable, crystalline form of Compound 1 that is neither hygroscopic nor deliquescent, and exhibits favorable thermal stability.

### Methods for Treating Conditions Mediated by URAT1

In another aspect, the present disclosure provides a method for treating a subject suffering from or at risk of a URAT1-mediated diseases or conditions. In one embodiment, the present disclosure provides a method for treating a subject suffering from or at risk of diseases or conditions associated with abnormally high expression of URAT1. In one embodiment, the present disclosure provides a method for treating a subject suffering from or at risk of diseases or conditions associated with chronically elevated serum uric acid levels. In one embodiment, the present disclosure provides a method for treating a subject suffering from or at risk of diseases or conditions associated with disorder of purine metabolism. In one embodiment, the present disclosure provides a method for treating a subject suffering from or at risk of diseases or conditions associated with insufficient renal elimination of uric acid. In one embodiment, the present disclosure provides a method for treating a subject suffering from or at risk of diseases or conditions associated with hyperuricemia. In one embodiment, the present disclosure provides a method for treating a subject suffering from or at risk of diseases or conditions associated with gout. The method includes administering to the subject an effective amount of Compound I Form 1. Compound I Form 2, Compound I Form 3, Compound I Form 4, Compound I Form 5A, Compound I Form 5B, Compound I Form 5C, Compound I Form 5D, Compound I Form 6, Compound I Form 7, Compound I Form 8, Compound I Form 9, Compound I Form 10, Compound I Form 11, or Compound I Form 12, as described herein, or a composition thereof. In certain embodiments, the method involves administering to the subject an effective amount of any one or more solid, crystalline, or polymorphs of Compound I as described herein in combination with one or more other therapies for the disease or condition.

In some embodiments, the disclosure provides a method for inhibiting URAT1. The method includes contacting Compound I Form 1, Compound I Form 2, Compound I Form 3, Compound I Form 4, Compound I Form 5A, Compound I Form 5B, Compound I Form 5C, Compound I Form 5D, Compound I Form 6, Compound I Form 7, Compound I Form 8, Compound I Form 9, Compound I Form 10, Compound I Form 11, or Compound I Form 12 as described herein, or a composition thereof with a cell or URAT1 either in vitro or in vivo.

In certain embodiments, the disclosure provides use of Compound I Form 1, Compound I Form 2, Compound I Form 3, Compound I Form 4, Compound I Form 5A, Compound I Form 5B, Compound I Form 5C, Compound I Form 5D, Compound I Form 6, Compound I Form 7, Compound I Form 8, Compound I Form 9, Compound I Form 10, Compound I Form 11, or Compound I Form 12, as described herein, or a composition thereof in the manufacture of a medicament for the treatment of a disease or condition as described herein. In other embodiments, the disclosure provides Compound I Form 1, Compound I Form 2, Compound I Form 3, Compound I Form 4, Compound I Form 5A, Compound I Form 5B, Compound I Form 5C, Compound I Form 5D, Compound I Form 6, Compound I Form 7, Compound I Form 8, Compound I Form 9, Compound I Form 10, Compound I Form 11, or Compound I Form 12, for use in treating a disease or condition as described herein.

In some embodiments, compositions are provided that include a therapeutically effective amount of any one or more solid, crystalline, or polymorphs of Compound I as described herein and at least one pharmaceutically acceptable carrier, excipient, and/or diluent, including combinations of any two or more any one or more solid, crystalline, or polymorphs of Compound I as described herein. In certain embodiments, the composition can include any one or more solid, crystalline, or polymorphs of Compound I as described herein along with one or more compounds that are therapeutically effective for the same disease indication. In one aspect, the composition includes any one or more solid, crystalline, or polymorphs of Compound I as described herein along with one or more compounds that are therapeutically effective for the same disease indication, wherein the compounds have a synergistic effect on the disease indication. In one embodiment, the composition includes any one or more solid, crystalline, or polymorphs of Compound I as described herein and one or more other compounds that are effective in treating gout or hyperuricemia, further wherein the compounds are synergistically effective in treating gout or hyperuricemia. The compounds can be administered simultaneously or sequentially.

In one embodiment, the disclosure provides methods for treating a disease or condition mediated by URAT1, by administering to the subject an effective amount of a composition including any one or more solid, crystalline, or polymorphs of Compound I as described herein in combination with one or more other suitable therapies as described herein for treating the disease.

### Kit

In another aspect, the disclosure provides kits or containers that include any solid, crystalline, or polymorphs of Compound I, or a pharmaceutically acceptable salt thereof, or a composition thereof as described herein. In some embodiments, the solid, crystalline, or polymorphs of Compound I or composition is packaged, e.g., in a vial, bottle, flask, which may be further packaged, e.g., within a box, envelope, or bag; the solid, crystalline, or polymorphs of Compound I or composition is approved by the U.S. Food and Drug Administration or similar regulatory agency for administration to a mammal, e.g., a human; the solid, crystalline, or polymorphs of Compound I or composition is approved for administration to a mammal, e.g., a human, for a URAT1-mediated disease or condition; the disclosure kit or container may include written instructions for use and/or other indication that the solid, crystalline, or polymorphs of Compound I or composition is suitable or approved for administration to a mammal, e.g., a human, for a URAT1-mediated disease or condition; and the solid, crystalline, or polymorphs of Compound I or composition may be packaged in unit dose or single dose form, e.g., single dose pills, capsules, or the like.

### Examples

### Dynamic Vapor Sorption/Desorption (DVS)

Moisture sorption/desorption data were collected on a SMS DVS intrinsic Vapor Sorption Analyzer under a nitrogen purge. The DVS experiment generally includes a moisture sorption step and a moisture desorption step. The experiment is deemed to have completed when the sample mass does not change over time. In other words, the sample is considered to have reached an equilibrium between moisture sorption and desorption under the relative humidity when dm/dt≤0.01%. The sample was maintained at the temperature of 25° C. with relative humidity varied from 0% to 95%, and back to 0% at an increment of 5% RH each step.

### Coulometric Karl-Fischer Analysis (KF)

Coulometric Karl Fischer (KF) analysis for water determination was performed using a Metrohm 787 KFKarl Fischer titrator.

### Differential Scanning Calorimetry (DSC)

DSC was performed using a Mettler Toledo DSC1differential scanning calorimeter. In a typical experiment, around 1 to 5 grams of sample was weighed into a closed aluminum crucible with a needle hole on the cap. The sample was scanned from 30° C. to 300° C. at a rate of 20° C./min under nitrogen protection.

### Thermogravimetric Analysis (TGA)

TG analyses were performed using a PerkinElmer Pyris 1 TGAthermogravimetric analyzer. In a typical experiment, around 5 grams of sample was weighed into the crucible under nitrogen protection. The sample was scanned at a temperature profile from 30° C. to 400° C.′ at the rate of 20° C./min. The result was calibrated against a blank background curve.

### Nuclear Magnetic Resonance Analysis (NMR)

Proton NMR was acquired using a Bruker AVANCE III 400 MHz instrument. In a typical experiment, the sample was prepared using about 3 mg of sample dissolved in about 0.5 mL deuterated dimethyl sulfoxide.

### High-Performance Liquid Chromatography (HPLC)

HPLC was acquired using Agilent 1260 instrument.

### X-Ray Powder Diffraction (XRPD)

X-ray powder diffraction patterns for Forms 1-12 were obtained using a Shimadzu XRD-6000equipped with a Cu Kα radiation source (λ=1.54056 Å), operating at a minimum supply of 40 kV and 30 mA. Data was collected for 2-Theta from 5 to 50 degrees at the speed of 5 degrees per minute.

Peaks identified here and throughout the disclosure are usually the more intense reflections in the powder patterns to avoid uncertainty due to the potential preferred orientation and particle statistic issues. Some of the peaks can be used to differentiate one crystalline polymorph from another crystalline polymorph. Some of these peaks may be unique, for example, are present in one crystalline polymorph of a compound but none of the other known crystalline polymorphs of the compound within ±0.2° 2θ. However, not all crystalline polymorphs of a compound necessarily have such unique peaks. In this case, multiple peaks may be used for identification purposes.

### Example 1. Synthesis of Compound I

Compound I was synthesized according to the synthetic scheme described below:

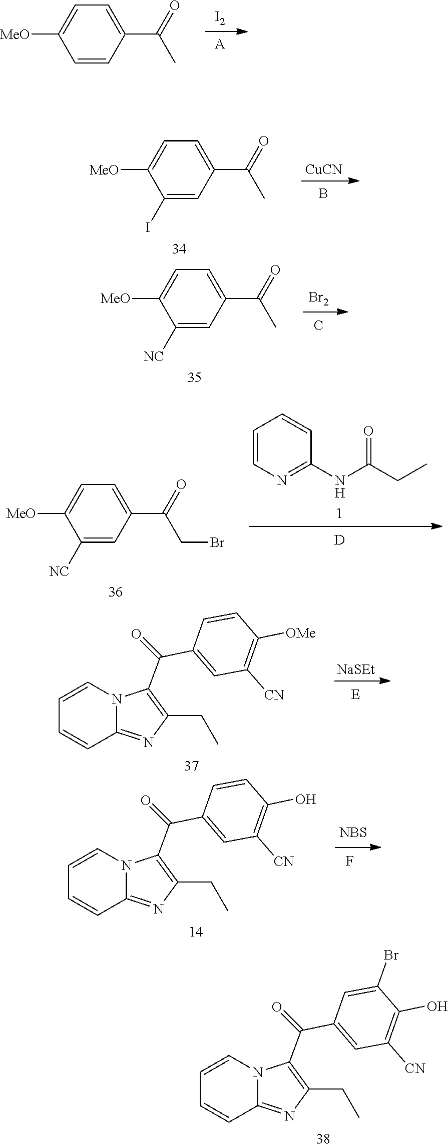

Step A: 1-(4-Methoxyphenyl) ethanone (44 g, 293 mmol) was added into a mixture of 1-chloromethyl-4-fluoro-1,4-diazoniabicyclo[2.2.2]octane bis(tetrafluoroborate) (104 g, 294 mmol) and iodine (38.6 g, 152 mmol) in acetonitrile (440 mL) in an ice-water bath. The reaction mixture was warmed to room temperature and stirred overnight. To the mixture was added water (1350 mL). The precipitates formed were collected by filtration, washed with water and dried to give 1-(3-iodo-4-methoxyphenyl) ethanone (34) (70 g) with 86.5% yield.

Step B: A mixture of compound 34 (70.0 g, 254 mmol) and cuprous cyanide (34.0 g, 380 mmol) in DMF (400 mL) was stirred at 130° C. overnight. The reaction mixture was cooled to room temperature and filtered through a celite pad. To the filtrate was added water (1600 mL), and the mixture was extracted with ethyl acetate (800 mL×3). The combined organic layer was washed with water (40 mL×2) and brine (400 mL), dried over anhydrous sodium sulfate, filtered, and concentrated under vacuum to give 5-acetyl-2-methoxybenzonitrile (35) (50.0 g). The crude product was used directly in the next step without further purification.

Step C: To a solution of crude compound 35 (45.0 g) in methanol (250 mL) was added bromine (49.0 g, 307 mmol) in methanol (50 mL), and the resulting mixture was stirred at room temperature overnight. To the mixture was added water (900 mL) and the precipitate were collected by filtration, washed with water and dried to give 5-(2-bromoacetyl)-2-methoxybenzonitrile (36) (41.0 g). The total yield of steps B and C was 70.6%.

Step D: A mixture of compound 36 (41.0 g, 161 mmol) and compound 1 (24.0 g, 161 mmol) in toluene (600 mL) was stirred at reflux for 48 h. The reaction mixture was cooled to room temperature, diluted with water (400 mL), adjusted to pH 7-8 with saturated sodium bicarbonate, and extracted with dichloromethane (600 mL×3). The combined organic layer was dried over anhydrous sodium sulfate and concentrated under vacuum. The residue was purified by flash column chromatography on silica gel (eluted with ethyl acetate/petroleum ether=1:30-2:1) to afford 5-[(2-ethylimidazo[1,2-a]pyridin-3-yl) carbonyl]-2-methoxybenzonitrile (37) (25.7 g) with 52.3% yield.

Step E: Sodium hydride (60% dispersion in mineral oil, 4.8 g, 120 mmol) was added portionwise to a solution of ethanethiol (8.4 mL) in THF (30 mL). The reaction mixture was stirred for about 5 minutes and filtered. The cake was added into a solution of compound 37 (9.0 g, 29.5 mmol) in DMF (25 mL). The resulting mixture was stirred at 60° C. for 2 h, cooled to room temperature, and filtered through a celite pad. To the filtrate was added water (100 mL), and the mixture was adjusted to pH 5-6 with 2 M aqueous citric acid. The precipitates formed were collected by filtration, washed with water, and dried. The cake was crystallized from acetonitrile to give 5-[(2-ethylimidazo[1,2-a]pyridin-3-yl) carbonyl]-2-hydroxybenzonitrile (14) (7.2 g) with 83.8% yield.

Step F: To a solution of compound 14 (7.2 g, 24.7 mmol) in DMF (70 mL) was added N-bromosuccinimide (5.28 g. 29.7 mmol) portionwise. After addition, the reaction mixture was stirred for another 1 h and diluted with water (210 mL). The precipitates were collected by filtration, washed with water and dried, to give 3-bromo-5-[(2-ethylimidazo[1,2-a]pyridin-3-yl) carbonyl]-2-hydroxybenzonitrile (38) (7.0 g) with 76.8% yield. ¹H NMR (DMSO-d6, 300 MHz) δ 9.01 (d, J=6.9 Hz, 1H), 8.02 (s, 1H), 7.83 (s, 1H), 7.78-7.75 (m, 1H), 7.65-7.59 (m, 1H), 7.22-7.17 (m, 1H), 2.58-2.50 (m, 2H), 1.19 (t, J=7.2 Hz, 3H). MS (EI, m/z): 368.0 [M-H].

### Example 2. Compound I Form 1

The product prepared from Example 1 was purified and crystalized from acetonitrile. The crystal received was characterized with XRPD and designated as Compound I Form 1. The redacted XRPD of Compound I Form 1 has been described with respect to FIG. 1 above. Moreover, the TGA and DSC of Compound I Form 1 have been described above with respect to FIGS. 2 and 3, respectively.

A heating study was conducted on a fresh sample of Form 1. The sample was heated to 140° C. with a TGA instrument and maintained for 30 minutes. XRPD pattern of the treated sample did not show any peaks, consistent with an amorphous form. This suggests that the endothermic peak on FIG. 3 within onset at about 140° C. was a melting peak of Form 1.

The moisture content of Form 1 was 8.8% as determined by KF method. Accordingly, each mole of the compound I in Form I was estimated to be associated with about 2 moles of water. DVS results showed Form 1 to be a hydrate. It rapidly absorbs moisture of about 3.4% by weight when the relative humidity was cycled from 0% RH to 5% RH, followed by a slower moisture sorption.

Form 1 was further characterized with ¹H-NMR, which shows peaks at 9.2 ppm, 8.2 ppm, 8.0 ppm, 7.8 ppm, 7.3 ppm, 4.3 ppm, 2.5 ppm, and 1.2 ppm.

In some embodiments. Form 1 prepared from wet acetonitrile, such as acetonitrile having a water content of about 0.5%, provided Form 1 in satisfactory crystalline quality.

### Solubility

Solubility of Form 1 was tested visually. About 5 mg of Form I was accurately weighed into a glass vial of 5 ml. Small amounts of solvent was added gradually into the vial until the compound was dissolved or when the total amount added reached 5 ml. The cumulative volume of the solvent was recorded. Calculated solubility from the experiment was shown in Table 1.

TABLE 1
Visual solubility of Form 1 in common solvents

| Solvent | Visual solubility(mg/ml) |
|---|---|
| Water (H₂O) | <1.0 |
| Methanol (MeOH) | <1.0 |
| Ethanol (EtOH) | <1.0 |
| Acetone | <1.0 |
| Tetrahydrofuran (THF) | 2.5~3.3 |
| Acetonitrile (ACN) | <1.0 |
| Ethyl acetate (EtAc) | <1.0 |
| Heptane | <1.0 |
| Dimethylsulfoxide (DMSO) | >25 |
| Dimethylformamide (DMF) | >25 |
| Isopropyl alcohol | <1.0 |
| Methyl ethyl ketone (MEK) | <1.0 |
| Dichloromethane (DCM) | <1.0 |
| Toluene | <1.0 |
| Methanol:water = 3:1 | <1.0 |
| Acetone:water = 3:1 | <1.0 |

As illustrated in Table 1, Form 1 was insoluble in water, and in organic solvents such as methanol, ethanol, acetone, acetonitrile, ethyl acetate, n-heptane, isopropanol, methyl ethyl ketone, dichloromethane, and toluene etc. It can be dissolved in tetrahydrofuran (THF) at a concentration of about 3 mg/ml, and has large solubility in dimethylsulfoxide (DMSO) and dimethylformamide (DMF) with a concentration of over 25 mg/ml.

### Heating Study

About 150 mg of Form 1 was heated to 110° C. and maintained for about 10 minutes. The sample color changed from light yellow to dark yellow. The darker sample was evaluated with XRPD. The sample color changed back to light yellow at the conclusion of the XRPD evaluation, which was similar to the color before the heating treatment. The same sample was further evaluated with TGA, which showed significant weight loss between room temperature and 85° C. Another sample of Form 1 was heated in an oven at 100° C. for 20 minutes and subsequently analyzed for the moisture content using KF method. The moisture content of heated Form 1 was 6.8%. It is thus concluded that Form 1 may lose water upon heating. The product is unstable and will rapidly absorb water.

### Example 3: Polymorph Screening

Compound I Form 1 was used as a starting material for polymorph screening. Several methods were used as described in detail below.

### Slurry

About 40 mg of Form 1 was weighed out into a respective 5 ml glass vial, and mixed with about 2 ml of various solvent as indicated in Table 2. All samples were kept stirring at room temperature or at 50° C. (as indicated in Table 2) for 24 hours. A suspension was formed. The wet solid was then isolated by centrifugation, and further dried in vacuum (40° C., −0.09MP). The dry solid was analyzed by XRPD, and the identification of crystalline forms are presented in Table 2.

### Anti-Solvent Method

About 50 mg of Form 1 was weighed out into a 40 ml glass bottle, and dissolved with 1 ml DMF. An anti-solvent was then added in dropwise until sufficient amount of solid precipitated out, or until 10 mL of anti-solvent had been added. When water was used as the anti-solvent, the solution became turbid after 1 ml of anti-solvent was added. However, a total of 3 ml was added. When methanol, acetone, or ethylacetate was used as the anti-solvent, no precipitation was observed after 10 ml of anti-solvent was added. All samples were kept stirring at room temperature for 24 hours. The wet solid precipitation was isolated by centrifugation, dried in vacuum (40° C., −0.09MP), and analyzed with XRPD. The identification of crystalline forms are presented in Table 2.

TABLE 2
Polymorph Screening

| | Slurry at Room | Slurry at | Anti- |
|---|---|---|---|
| Solvent | Temperature | 50° C. | solvent |
| Water | Form 1 | Form 1 | Form 1 |
| Methanol(MeOH) | Form 1 | Form 1 | Form 4 |
| Acetone | Form 2 | Form 2 | Form 2 |
| Acetonitrile(ACN) | Form 3 | Form 2 | — |
| Ethyl acetate(EtOAc) | Mixed crystal form | Form 2 | Form 2 |
| Methylethylketone(MEK) | Form 1 | Form 2 | — |
| Dichloromethane(DCM) | Form 1 | — | — |
| Toluene | Form 1 | Form 1 | — |
| Heptane | Form 1 | Form 1 | — |
| MeOH:water(3:1) | Form 1 | Form 1 | — |
| Acetone:water(3:1) | Form 1 | Form 1 | — |
| Tetrahydrofuran (THF) | Form 1 | Form 3 | — |

### Solvent Evaporation

About 25 mg of Form 1 was weighed out into a 40 ml glass bottle, and dissolved in 8 ml THF with sonication. The bottles were then placed in a fume hood, uncapped, such that the solvent may escape at room temperature. After 24 hours, any residual solvent was further dried by blowing nitrogen over the sample. The solid was collected and analyzed by XPRD, which shows a pattern consistent with Form 1, albeit with lower crystal quality.

### Grinding Method

About 80˜100 mg of Form 1 was weighed out into an agate mortar, and soaked with a small amount of acetone or THF. The sample was ground until the solvent disappeared. Another portion of the same solvent was added and the samples were further ground until the solvent again disappeared. This operation was repeated several times until the total grinding time reached about 5 minutes. The final sample was analyzed by XRPD. Both samples treated with THF and acetone showed XRPD that were consistent with Form 1.

### New Crystalline Forms Prepared

As described above, Form 2 may be prepared from acetone, acetonitrile, ethyl acetate, or methylethylketone. For example, Form 2 may be prepared by room temperature slurry method and anti-solvent method in acetone system: by slurry method at 50° C. and anti-solvent method in ethyl acetate system; or by slurry method at 50° C. in methylethylketone and acetonitrile system.

TGA and DSC of Form 2 were described above with respect to FIGS. 5 and 6. KF analysis revealed that Form 2 obtained from slurry in acetone included a moisture content of about 0.4%. DVS analysis showed that Form 2 had low hygroscopicity at a relative humidity less than 80%. However, Form 2 immediately absorbed about 3.8% water at when relative humidity reached 85% RH, and further absorbed more water as relative humidity increased. As the relative humidity decreased back to 0% in the desorption process, about 3.4% water remained within the sample. XRPD on the sample following DVS analysis demonstrated that the sample had been converted into Form 1. This also confirms that Form 1 is a hydrate.

Form 3 may be obtained from acetonitrile slurry at room temperature and from THE slurry at 50° C., as shown by XRPD patterns. The XRPD pattern was described above with respect to FIG. 7; TGA and DSC analysis of the sample prepared from THE slurry at 50° C. were described above with respect to FIGS. 8 and 9. Form 3 is contemplated to be a relatively unstable solvate.

Form 4 may be prepared from DMF solution using methanol as the antisolvent. The XRPD pattern was described above with respect to FIG. 10; TGA and DSC for Form 4 were described above with respect to FIGS. 11 and 12.

Additional observations and data for the forms are presented in Table 3.

TABLE 1
Physical characterization results of the new crystal forms

| Crystal | | | DSC(° C.) | DVS |
|---|---|---|---|---|
| Form | Preparation | TGA | (Onset~Peak~Endset) | (80% RH) |
| Form 1 | N/A | 4.74% | endothermic peak 1→solvent | 5.32% |
| | | (RT~120.0° C.) | (92.89~102.30~106.94) | (Losecrystalline |
| | | 3.84% | endothermic peak 2→solvent | water when the |
| | | (120.0° C.~180.0° C.) | (137.88~158.51~167.15) | relative humidity |
| | | | exothermic peak 1→decomposition | is less than 5%) |
| | | | (252.64~273.66~290.40) | |
| Form 2 | Slurry in | 0.03% | endothermic peak 1→melting | 0.40% |
| | acetone at | (RT~120.0° C.) | (252.97~256.01~259.95) | (Quickly absorbs |
| | 50° C. | | exothermic peak 1→decomposition | water when the |
| | | | (265.40~275.85~286.25) | relative humidity |
| | | | | is more than |
| | | | | 80%) |
| Form 3 | Slurry in THF | 0.22% | endothermic peak 1→solvent | Not tested |
| | at 50° C. | (RT~120.0° C.) | (162.84~171.38~175.17) | |
| | | 4.51% | endothermic peak 2→melting | |
| | | (120.0° C.~214.1° C. | (248.01~251.10~258.00) | |
| Form 4 | Anti-solvent | 2.44% | endothermic peak 1→solvent | Not tested |
| | (DMF/MeOH) | (RT~120.0° C.) | (84.75~102.20~109.88) | |
| | | 5.78% | endothermic peak 2→solvent | |
| | | (120.0° C.~171.7° C.) | (124.75~149.45~163.05) | |
| | | | exothermic peak 1→crystal transition | |
| | | | (172.72~177.83~182.11) | |
| | | | endothermic peak 3→melting | |

### Example 4: Salt Screening

### Small Scale

Compound I Form 1 (80˜100 mg) was prepared into eight samples with either THF or acetones the solvent (as indicated below in Table 4) at the ratio of about 1.0 mL solvent per 10 mg Compound I Form 1. The samples whereto form uniform suspensions. Subsequently, hydrochloric acid, sulfuric acid, nitric acid, maleic acid, methanesulfonic acid, benzenesulfonic acid, potassium hydroxide, and sodium hydroxide (collectively referred to as the counter-ions) were each added into a respective glass vials at a molar ratio of 1:1.05 (Compound I: counter-ions) unless otherwise noted. All samples were further magnetically stirred overnight at room temperature. The salts were collected either by centrifugation or by solvent evaporation and further dried in vacuum at 40° C. before being analyzed with XRPD. The experimental results are summarized in Table 4. Blank control samples were used to confirm that similar treatment with the exception of introduction of counter-ions did not result in any change in crystalline forms.

TABLE 4
Salt Screening

| | | Volume | | | |
|---|---|---|---|---|---|
| Counter-ion | | of | | | |
| (Conc. | Solvent | counter- | | | |
| solvent) | (ml) | ions | | Description | XRPD |
| Hydrochloric acid | THF | 114 | μl | The yellow suspension transformed into white | Form |
| (2 mol/L in water) | (8.0) | | | suspension upon addition of the counter-ion. | 5A |
| Maleic acid | THF | 228 | μl | The yellow suspensiondidnot change upon | Form |
| (1 mol/L in | (8.0) | | | addition of the counter-ion. Small amount of | 10 |
| methanol) | | | | solid received after stirring for 24 hours. The | |
| | | | | supernatant solution was evaporated for further | |
| | | | | analysis. | |
| Methanesulfonic | THF | 228 | μl | The yellow suspensiondid not change upon | Form |
| acid | (8.0) | | | addition of the counter-ion. White suspension | 6 |
| (1 mol/L in water) | | | | was obtained following stirring for 24 hours. | |
| benzenesulfonic | THF | 228 | μl | The yellow suspensiondid not change upon | Form |
| acid | (8.0) | | | addition of the counter-ion. White suspension | 1 |
| (1 mol/L in water) | | | | was obtained following stirring for 24 hours. | |
| Sulfuric acid | THF | 114 | μl | The yellow suspensiondid not change upon | Form |
| (2 mol/L/ in water) | (8.0) | | | addition of the counter-ion. White suspension | 8 |
| | | | | was obtained following stirring for 24 hours. | |
| Nitric acid | THF | 114 | μl | The color of the yellow suspension deepened | Form |
| (2 mol/L in water) | (8.0) | | | upon addition of the counter-ion. Yellow | 9 |
| | | | | suspension was obtained following stirring for | |
| | | | | 24 hours. | |
| Sodium hydroxide | THF | 30 | μl | The yellow suspensiondid not change upon | Form |
| (5 mol/L in water)^{a} | (5.0) | | | addition of the counter-ion. A solution in light | 12 |
| | | | | yellow was received after stirring for 24 hours. | |
| | | | | The solvent was evaporated and the solid thus | |
| | | | | received was subject to further analysis. | |
| Potassium | THF | 46 | μl | The yellow suspensionchanged into white | Form |
| hydroxide | (8.0) | | | suspension upon addition of the counter-ion. | 11 |
| (5 mol/L in water) | | | | | |
| Hydrochloric Acid | Acetone | 114 | μl | The yellow suspensionchanged into white | Form |
| (2 mol/L in water) | (8.0) | | | suspension upon addition of the counter-ion. | 5B |
| Maleic acid | Acetone | 228 | μl | The yellow suspensiondid not change upon | Form |
| (1 mol/L in | (8.0) | | | addition of the counter-ion. A suspension in light | 1 |
| methanol) | | | | yellow was received after being stirred for 24 | |
| | | | | hours. | |
| Methanesulfonic | Acetone | 228 | μl | The yellow suspensiondid not change upon | Form |
| acid | (8.0) | | | addition of the counter-ion. A suspension in | 6 |
| (1 mol/L in water) | | | | semi-white was received after being stirred for | |
| | | | | 24 hours. | |
| Benzenesulfonic | Acetone | 228 | μl | The yellow suspensiongradually changed into | Form |
| acid | (5.0) | | | white suspension upon addition of the counter- | 7 |
| (1 mol/L in | | | | ion. A suspension in white was received after | |
| water)^{a} | | | | being stirred for 24 hours. | |
| Sulfonic acid | Acetone | 114 | μl | The yellow suspension turned into a clear | Form |
| (2 mol/L in water) | (8.0) | | | solution upon addition of the counter-ion. | 8 |
| | | | | A suspension in white was received after being | |
| | | | | stirred for 24 hours. | |
| Nitric acid | Acetone | 114 | μl | The yellow suspensionturned into dark yellow | Form |
| (2 mol/L in water) | (8.0) | | | in color upon addition of the counter-ion. A | 9 |
| | | | | suspension in light yellow was received after | |
| | | | | being stirred for 24 hours. | |
| Sodium hydroxide | Acetone | 30 | μl | The yellow suspensiondid not change upon | |
| (5 mol/L in | (5.0) | | | addition of the counter-ion. A solution in light | |
| water)^{a,b} | | | | yellow was received after being stirred for 24 | |
| | | | | hours. The acetone in the sample could not be | |
| | | | | satisfactorily removed. Thus the sample was not | |
| | | | | evaluated with XRPD. | |
| Sodium hydroxide | Acetone | 30 | μl | The yellow suspensionturned into a white | Form |
| (5 mol/L in | (2.0) | | | suspension upon addition of the counter-ion. A | 12 |
| water)^{a,b} | | | | solution in semi-white was received after being | |
| | | | | stirred for 24 hours. | |
| Potassium | Acetone | 46 | μl | The yellow suspensionturned into a white | Form |
| hydroxide | (8.0) | | | suspension upon addition of the counter-ion. | 11 |
| (5 mol/L in water) | | | | | |
| HCl | Methanol | 142 | μl | The yellow suspensionturned into a clear | Form |
| (2 mol/L in water)^{c} | 10 ml | | | solution upon addition of the counter-ion. A | 5C |
| | | | | suspension in light yellow was received after | |
| | | | | being stirred for 24 hours. The solvent was | |
| | | | | evaporated to provide the sample for analysis. | |
| HCl | Ethanol | 71 | μl | The color of the yellow suspensiondeepened | Form |
| (2 mol/L in | 2 ml | | | upon addition of the counter-ion. A solution in | 5D |
| methanol)^{a} | | | | yellow was received after being stirred for 24 | |
| | | | | hours. | |

^{a}About 50 mg of Compound I was used.
^{b}The ratio of Compound I:acid was 1:1.1.
^{c}About 100 mg of Compound I was used.

As illustrated, in the THF system, Compound I forms salts with all acids except benzenesulfonic acid; in the acetone system, it forms salt with all acids except maleic acid. Corresponding salts obtained from THF and from acetone have matched XRPD results, thus are of the same crystalline form. The XRPD patterns for the new crystalline forms have been described above. Further observations and data of Form 1 and the salt forms are presented in Table 5 below.

TABLE 5
Characterizations of Form 1 and its Salt Forms

| Salt | | | DSC (° C.) | DVS | NMR | |
|---|---|---|---|---|---|---|
| Forms | Preparation | TGA | (Onset~peak~endset) | (85% RH) | (Molar ratio) | Conclusion |
| Form | — | 4.74% | endothermic | 5.68% | N/A | N/A |
| 1 | | (30.0° C.~120.0° C.) | peak1→solvent | (would | | |
| | | 3.84% | (92.89~102.30~106.94) | lose | | |
| | | (120.0° C.~180° C.) | endothermic | crystalline | | |
| | | | peak2→melting | H₂O in <5% | | |
| | | | (137.88~158.51~167.15) | RH) | | |
| | | | exothermic | | | |
| | | | peak1→decomposition | | | |
| | | | (252.64~273.66~290.40) | | | |
| Form | HCl/THF | 1.28% | endothermic | — | Contain | Solvate |
| 5A | | (30.0° C.~120.0° C.) | peak1→solvent | | THF | |
| | | 14.16% | (126.0~139.7~163.6) | | | |
| | | (120.0° C.~190.1° C.) | endothermic | | | |
| | | | peak2→melting | | | |
| | | | (222.1~245.5~252.4) | | | |
| | | | exothermic | | | |
| | | | peak1→decomposition | | | |
| | | | (257.6~266.4~282.1) | | | |
| Form | HCl/ | 15.14% | endothermic | — | Contain | Solvate |
| 5B | Acetone | (20.4° C.~180.0° C.) | peak1→solvent | | acetone | |
| | | | (102.2~111.3~120.7) | | | |
| | | | endothermic | | | |
| | | | peak2→melting | | | |
| | | | (233.0~249.2~254.3) | | | |
| | | | exothermic | | | |
| | | | peak1→decomposition | | | |
| | | | (258.5~266.9~282.6) | | | |
| Form | HCl/ | 1.295% | endothermic | — | — | Solvate |
| 5C | Methanol | (20.62° C.~120.0° C.) | peak1→solvent | | | |
| | | 4.575% | (153.5~164.4~172.0) | | | |
| | | (120.0° C.~236.2° C.) | endothermic | | | |
| | | | peak2→melting | | | |
| | | | (184.0~202.5~212.8) | | | |
| | | | exothermic | | | |
| | | | peak1→decomposition | | | |
| | | | (254.3~271.9~286.9) | | | |
| Form | HCl/ | 0.309% | endothermic | 2.14% | No | anhydrous |
| 5D | Ethanol | (20.87° C.~120.0° C.) | peak1→solvent | | ethanol | substance |
| | | 3.979% | (181.7~197.2~210.5) | | residual | |
| | | (120.0° C.~210.2° C.) | exothermic | | | |
| | | | peak1→decomposition | | | |
| | | | (253.1~273.7~289.1) | | | |
| Form6 | Mesylate/ | 3.71% | endothermic | 0.35% | No | Hydrate |
| | Acetone | (30.7° C.~128.2° C.) | peak1→crystal water | | acetone | |
| | | | (128.7~151.4~158.1) | | residual | |
| | | | endothermic | | (1:0.96) | |
| | | | peak2→melting | | | |
| | | | (256.7~259.7~259.8) | | | |
| | | | exothermic | | | |
| | | | peak1→decomposition | | | |
| | | | (264.2~272.0~288.4) | | | |
| Form | Besylate/ | 0.04% | endothermic | 1.93% | Contain | anhydrous |
| 7 | Acetone | (20.0° C.~120.0° C.) | peak1→melting | | benzenesulfonic | |
| | | | (254.8~259.2~262.6) | | acid | |
| | | | exothermic | | | |
| | | | peak1→decomposition | | | |
| | | | (264.3~275.6~291.6) | | | |
| Form | Sulfate/ | 0.10% | endothermic | 4.66% | No | anhydrous |
| 8 | Acetone | (30.0° C.~120.0° C.) | peak1→melting | | acetone | |
| | | | (238.2~242.9~247.0) | | residual | |
| Form | Nitrate/ | 0.32% | endothermic | 0.29% | No | anhydrous |
| 9 | Acetone | (20.2° C.~120.0° C.) | peak1→melting | | acetone | |
| | | 8.09% | (164.4~172.4~177.5) | | residual | |
| | | (120.0° C.~215.7° C.) | exothermic | | | |
| | | | peak1→decomposition | | | |
| | | | (244.7~270.6~289.2) | | | |
| Form | Potassium | 0.17% | endothermic | 6.18% | No | anhydrous |
| 11 | Salt/ | (30.0° C.~120.0° C.) | peak1→melting and | | acetone | |
| | Acetone | | decomposition | | residual | |
| | | | (368.0~372.7~377.5) | | | |
| Form | Maleate/ | 0.17% | endothermic | 2.37% | 1:0.56 | anhydrous |
| 10 | THF | (30.0° C.~120.0° C.) | peak1→melting and | | | |
| | | 18.41% | decomposition | | | |
| | | (120° C.~242.05° C.) | (167.3~177.5~184.2) | | | |
| Form | Sodium | 11.58% | endothermic | — | Not | Containing |
| 12 | Salt/ | (21.28° C.~150.0° C.) | peak1→solvent | | analyzed | a lot of |
| | THF | 2.746% | (80.5~94.8~108.4) | | | water |
| | | (150.0° C.~226.9° C.) | endothermic | | | |
| | | | peak2→solvent | | | |
| | | | (127.8~142.4~152.2) | | | |
| | | | exothermic | | | |
| | | | peak1→decomposition | | | |
| | | | (373.4~381.4~387.0) | | | |
| Form | Sodium | 8.79% | endothermic | 8.07% | No | Containing |
| 12 | Salt/ | (20.90° C.~150.0° C.) | peak1→solvent | | acetone | a lot of |
| | Acetone | | (87.3~106.7~113.7) | | residual | H₂O |
| | | | endothermic | | | |
| | | | peak2→solvent | | | |
| | | | (116.1~136.6~164.7) | | | |

### Example 5: Scale-Up and Characterization of Salt Forms

The hydrochloric salt (Form 5D) was scaled up as follows. About 400 mg of Compound I Form 1 was weighed into a 40 ml glass bottle and mixed with about 10 ml of ethanol to form a light yellow suspension. Subsequently, a hydrochloric acid solution (2 mol/L in methanol) was introduced at the molar ratio of 1:1.05 (Compound I: counter-ion). The color of the suspension deepened after the addition. The sample was kept stirring at room temperature on a plate for 24 hours. The solid was collected by centrifugation and dried for 4 to 6 hours. The dried solids was each analyzed with XRPD. The ion ratio of Compound I to counter-ion in the hydrochloride salt was determined by HPLC-ELSD to be 1:0.50 and 1:0.88 in two separate batches. It is thus concluded that it is difficult to form hydrochloride salt with a stable molar ratio.

The mesylate (Form 6) was similarly scaled up with methanesulfonic acid (1 mol/L in water), although the color of the suspension changed from light yellow to white upon the addition of acid. The ion ratio of Compound I to counter-ion in the mesylate was determined by 1H-NMR to be 1:0.96. Thus the Compound could form a mesylate salt with an approximate molar ratio of 1:1.

The mesylate prepared from acetone was heated to 160° C. by TGA instrument and kept at 160° C. for 5 min to remove the solvent, and then analyzed by XRPD. The de-solvated mesylate was placed at high humidity condition (92.5% RH) overnight, and again analyzed with XRPD. The result was shown in FIG. 37. As illustrated, the crystal form of mesylate changed after the loss of solvent, but after moisture re-absorption in high humidity, it slowly returned to the initial crystal form. It was thus shown that the mesylate was a hydrate.

The sulfate (Form 8) was similarly scaled up with sulfuric acid (2 mol/L in water), although the light yellow suspension turned into a transparent solution upon the addition of acid. The ion ratio of Compound I to counter-ion in the sulfate was tested at Shanghai Metrology Institute to be 1:0.52, thus indicating each mole of Compound I is associated with about 0.5 mile of sulfuric acid in Form 8.

The potassium salt (Form 11) was similarly scaled up with potassium hydroxide (5 mol/L in water), although the light yellow suspension did not undergo any substantial change upon the addition of base. The ion ratio of Compound I to counter-ion in the potassium salt was also tested at Shanghai Metrology Institute to be 1:0.86, thus indicating salt formation was not complete.

Each of the scaled-up salts presented XRPD consistent to those obtained at the 80 mg˜100 mg scale.

### Example 6: Solubility of Form 1, Form 2, Form 5D, Form 6, Form 8, and Form 11

The solubility of Form 1, Form 2, Form 5D, Form 6, Form 8, and Form 11 was analyzed in water, 0.1NaqueousHCl, pH4.5 acetate buffer solution, pH6.8 phosphate buffer solution, simulated gastric fluid (SGF), Fasted State Simulated Intestinal Fluid (FaSSIF), and Fed State Simulated Intestinal Fluid (FeSSIF).

About 5 mg or 10 mg of sample was weighed out into each vial and mixed with about 5 ml of medium to form a solution. The target concentration for the potassium salt in water was 2 mg/ml; and the target concentration for all other forms were 1 mg/mL. All samples were kept shaking at 200 rounds per minute (rpm) under 37° C. for 24 hours. The pH value of these samples were determined after shaking for 24 hours. Subsequently, they were centrifuged at the speed of 12000 rpm for 2 mins. The supernatant was diluted with methanol as necessary, and the concentrations were determined by HPLC. When the solubility of potassium salt was tested in water, the supernatant was diluted 100 times, while no other samples were diluted. The HPLC analysis utilizes Agilent Eclipse XDB-C8 4.6*150 mm, 5 μm column at 40° C., a mobile phase of 0.1% TFA: ACN=70:30, an injection volume of 10 μl, a flow rate of 1.0 ml/min, a diluent of methanol, and a detection wavelength of 234 nm.

Form 1 and Form 2became homogeneous suspensions in all medium after being shaked for about 24 hours. Form 1 and Form 2 both had low solubilities in the buffer solution and bio-relevant medium. However, Form 2 did present higher solubility in several mediums, with the highest concentration at around 40 μg/ml.

Form 11 formed nearly clear solution in water, but formed homogeneous suspensions in all other media, similar to Form 1. The increase of solubility of potassium salt (Form 11) in water may be caused by the substantial increase of pH value. Meanwhile, Form 5D, Form 6, and Form 8 each became heterogeneous suspension with small particles after the same treatment, indicating reduced solubility in water than the free base form (Form 1).

The solubility of Form 1, Form 2, Form 5D, Form 6, Form 8, and Form 11 are presented in Table 6.

TABLE 6
Solubility of Form 1, its salts, and Form 2 (37° C.)

| Medium | Form 1 | Form 2 | Form 5D | Form 6 | Form 8 | Form 11 |
|---|---|---|---|---|---|---|
| Water | 9.4 | 20.0 | 0.8 | 0.4 | 0.5 | 1914.5 |
| 0.1N HCl | 5.0 | 3.8 | 8.3 | 4.6 | 5.3 | 6.8 |
| SGF | 2.9 | 3.0 | 7.4 | 3.3 | 3.5 | 5.8 |
| pH 4.5 | 1.0 | <1 | 1.3 | 0.5 | 0.6 | 1.7 |
| acetate | | | | | | |
| buffer | | | | | | |
| pH 6.8 | 19.8 | 24.1 | 47.7 | 25.7 | 17.9 | 31.0 |
| phosphate | | | | | | |
| buffer | | | | | | |
| FaSSIF | 16.6 | 41.8 | 55.8 | 45.8 | 14.5 | 28.4 |
| FeSSIF | 1.9 | 2.8 | 5.4 | 4.4 | 8.6 | 3.4 |

### Example 7: Stability of Forms

Slurry stability of Form 1, Form 5D, Form 6, and Form 8.

About 30 mg of Form land Form 5D, Form 6, and Form 8 were mixed with about 2.5 ml of purified water, and the mixtures were stirred for overnight at room temperature. The wet solids were isolated by centrifugation, and further dried in vacuum for 2.5 hours before being analyzed with XRPD. The XRPD results showed that each of Form 5D, Form 6, and Form 8 transformed into Form 1 after the experiment.

Solid state stability study of Form 1, Form 2, Form 6, Form 8, and Form 11

Form 1. Form 2, Form 6, Form 8, and Form 11 were each placed under following stress conditions: (1) in a closed vial at a temperature of about 60° C., and (2) in a closed vial at a temperature of about 40′° C. and a humidity of about 75% RH. The solids were analyzed with XRPD at 1 week and 2 weeks intervals. All forms presented XRPD patterns substantially similar to those received at the initial time, indicating their stability under these stress conditions.

Additionally, all forms were analyzed with HPLC for impurity at the 1 week and 2 weeks intervals. In a typical analysis, about 5 mg of forms were each dissolved in 10 ml of diluents with the aid of sonication for 2 minutes. THF was used as the diluent for Form 1, and methanol was used as the diluent for Form 6, Form 8, and Form 11. The HPLC analysis utilized Agilent Eclipse XDB-C18 4.6*150 mm. 3.5 μm at 30° C., a mobile phase of a gradient between 0.1% H₃PO₄ and acetonitrile (9:1 to 2:8 to 9:1), an injection volume of 10 μl, a flow rate of 1.0 ml/min, and a detection wavelength of 214 nm. The results revealed no significant change as compared to HPLC data received at the initial time, confirming that all forms have a good stability under these stress conditions.

Form 2 was further subject to the stability test in an open vial at 40° C. and a relative humidity of 75% RH. The XRPD analysis illustrates significant changes at the end of ninth day, suggesting that Form 2 should be stored away from moisture.

### Example 8: Suspensions for Animal Formulation

About 30 mg of Form 1, Form 2, and Form 5D were each added into a respective 40 ml glass bottle and mixed with 30 ml of 0.5% CMC-Na solution. The solids were dispersed uniformly in the solution with the aid of sonication. After that, the solids were isolated by centrifugation, dried at room temperature, and the dry solid analyzed with XRPD. The analysis showed that Form 2 transformed into Form 1 under the condition, while Form 1 and Form 5D did not change.

### Example 9: Pharmacokinetics study of Formsin Rats

Form 1, Form 2, and Form 5D were each evaluated via a single-dose pharmacokinetic study following oral and intravenous administration in SD rats. The results showed that the mean bioavailability for all the test articles were satisfactory at about 50%. Form 2 showed a higher dose exposure and bio-availability than Form 1, and is thus a desirable drug candidate for the formulation development. Form 1, Form 2, and Form 5Dwere evaluated by rat pharmacokinetics study.

### Materials and Instruments

Male SD rat was purchased from Shanghai Sippr B&K Laboratory Animals Co., Ltd. The weight was 180 g˜200 g.

### Preparation of Test Article

20% HP-β-CD was prepared from dissolving20 g of HP-β-CD (from Sigma) in 100 mL purified water. 0.5% CMC-Na was prepared from dissolving 1 g of CMC-Na (from Aladdin) in 200 mL purified water. The solutions were stored at 2-8 degree.

Group 1:4.95 mg (equivalent to 4.50 mg of anhydrous free base) of Form 1 was weighed out into a 20 ml bottle, and totally dissolved by 0.225 mL DMSO by sonication. After that, 4.275 mL of 20% HP-β-CD was added, mixed well by sonication. Finally, the pH value was adjusted to 7.0 by sodium hydroxide solution. The clear solution was obtained, and the concentration was 1 mg/mL.

Group 2:10.08 mg (equivalent to 9.164 mg of anhydrous free base) of Form 1 was weighed out into a 20 ml bottle, and then 9.164 mL of 0.5% CMC-Na was added. A white suspension was formed after sonicated by ultrasonic cell disruptor, and the concentration was 1 mg/mL.

Group 3:9.47 mg of Form 2 was weighed out into a 20 ml bottle, and then 9.470 mL of 0.5% CMC-Na was added. After sonicated, it was mixed well by homogenizer for 2 minutes. Finally, a white suspension was formed, and the concentration was 1 mg/mL.

Group 4:9.80 mg (equivalent to 8.909 mg of free base) of Form 5Dwas weighed out into a 20 mL bottle, and then 8.909 mL of 0.5% CMC-Na was added. After sonicated, it was mixed well by homogenizer for 2 minutes. Finally, a white suspension was formed, and the concentration was 1 mg/mL.

Group 5:1.94 mg (equivalent to 1.763 mg of anhydrous free base) of Form 1 was weighed out into a 20 ml bottle, and totally dissolved by 0.088 mL DMSO by sonication. After that, 1.675 mL of 20% HP-β-CD was added, mixed well by sonication. Finally, the pH value was adjusted to 7.0 by sodium hydroxide solution. The clear solution was obtained, and the concentration was 0.2 mg/mL.

### Drug Administration and Blood Sampling

The dose of intravenous administration was 1 mg/kg and 5 mg/kg, respectively. The oral dose was 10 mg/kg. Animals were fasted overnight before drug administration. After the drug administration, the rats were fed at the 4 hours.

About 150˜200 μL of blood was collected at the initial time (only intravenous), and at 0.25, 0.5, 1, 2, 4, 6, 8 and 24 h by jugular venipuncture blooding method. EDTA-K2 was selected as anticoagulant. Blood samples were centrifuged within one hour at 6000 rpm for 8 minutes (placed on wet ice before centrifugation). The supernatant was stored at −20° C. for LC-MS/MS analysis.

The computer program Microsoft Office Excel 2007 (Microsoft, USA) was used to process data and graphics. The pharmacokinetic parameters were calculated by WinNolin 6.4 software.

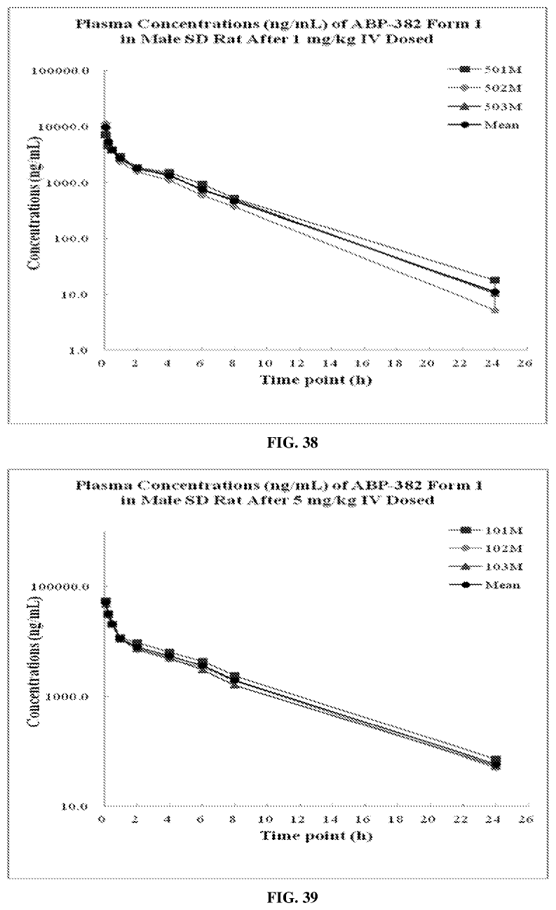

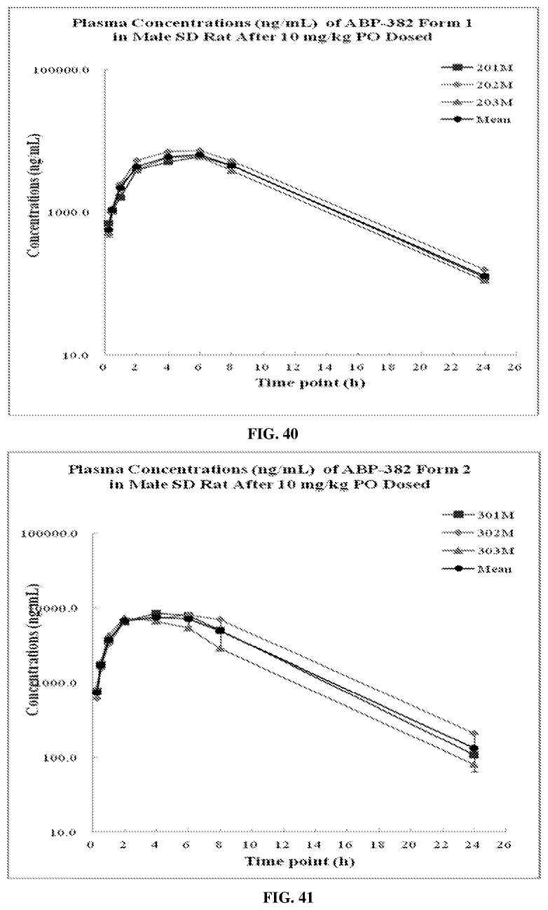

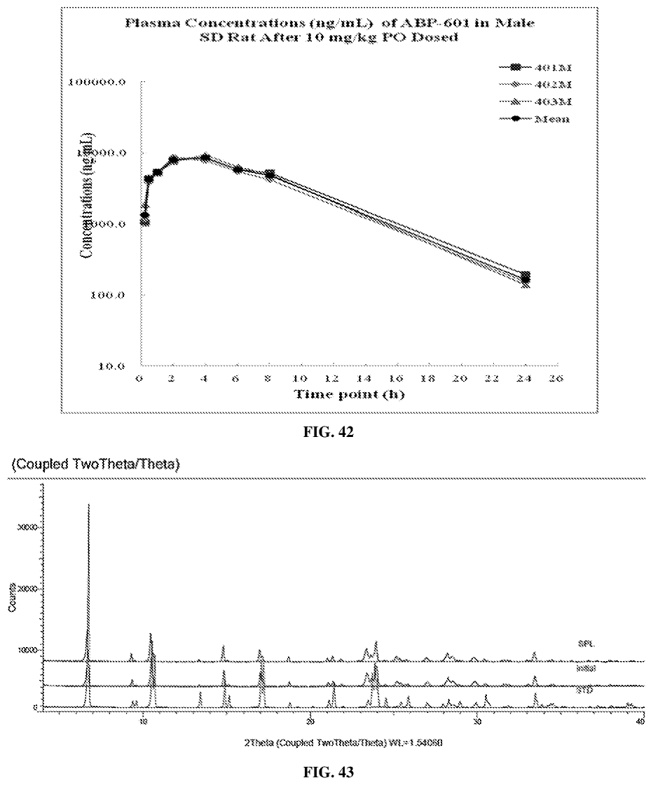

After intravenous and oral administration on male SD rat, the drug concentration in plasma at different time points was collected. FIG. 38 illustrates the plasma concentration—time curve of a single intravenous administration of 1 mg/kg for Compound I Form 1 in SD rats. FIG. 39 illustrates the plasma concentration-time curve of a single intravenous administration of 5 mg/kg for Compound I Form 1 in SD rats. FIG. 40 illustrates the plasma concentration-time curve of a single oral administration of 10 mg/kg for Compound I Form 1 in SD rats. FIG. 41 illustrates the plasma concentration-time curve of a single oral administration of 10 mg/kg for Compound I Form 2 in SD rats. FIG. 42 illustrates the plasma concentration-time curve of a single oral administration of 10 mg/kg for Compound I Form 5D in SD rats. The pharmacokinetic parameters for noncompartmental model are presented in Tables 7 to 11.

TABLE 7
Pharmacokinetic parameters of noncompartmental model following intravenous
administration of 1 mg/kg for Form 1 in male SD rats

| Parameters | 501 | 502 | 503 | Average | SD |
|---|---|---|---|---|---|
| HL_Lambda_z (T_{1/2}, hr) | 3.20 | 2.61 | 2.90 | 2.90 | 0.29 |
| Cₘₐₓ (ng/mL) | 7116.3 | 11256.4 | 10356.4 | 9576.4 | 2177.5 |
| AUCₗₐₛₜ (hr*ng/ml) | 18450.5 | 15448.2 | 18170.6 | 17356.4 | 1658.5 |
| AUC_{INF}_{_{—}}_{pred} (hr*ng/ml) | 18531.5 | 15467.7 | 18214.0 | 17404.4 | 1684.7 |
| MRTₗₐₛₜ (hr) | 3.99 | 3.28 | 3.68 | 3.65 | 0.36 |
| V_{z}_{_{—}}_{pred} (L/kg) | 0.25 | 0.24 | 0.23 | 0.24 | 0.01 |
| Cl_{_{—}}_{pred} (L/hr/kg) | 0.05 | 0.06 | 0.05 | 0.06 | 0.01 |
| λz Calculation Time Range (hr) | 6-24 | 4-24 | 6-24 | NA | NA |

TABLE 8
Pharmacokinetic parameters of noncompartmental model after following
administration of 5 mg/kg for Form 1 in male SD rats

| Parameters | 101 | 102 | 103 | Average | SD |
|---|---|---|---|---|---|
| HL_Lambda_z (T_{1/2}, hr) | 3.12 | 3.03 | 3.10 | 3.08 | 0.05 |
| Cₘₐₓ (ng/mL) | 55666.4 | 51717.5 | 46753.2 | 51379.0 | 4466.2 |
| AUCₗₐₛₜ (hr*ng/ml) | 90790.9 | 77235.7 | 72609.9 | 80212.2 | 9448.9 |
| AUC_{INF}_{_{—}}_{pred} (hr*ng/ml) | 91117.6 | 77458.7 | 72847.5 | 80474.6 | 9501.1 |
| MRTₗₐₛₜ (hr) | 3.65 | 3.40 | 3.28 | 3.44 | 0.19 |
| V_{z}_{_{—}}_{pred} (L/kg) | 0.25 | 0.28 | 0.31 | 0.28 | 0.03 |
| Cl_{_{—}}_{pred} (L/hr/kg) | 0.05 | 0.06 | 0.07 | 0.06 | 0.01 |
| λz Calculation Time Range (hr) | 1-24 | 4-24 | 2-24 | NA | NA |

TABLE 9
Pharmacokinetic parameters of noncompartmental model following
oral administration of 10 mg/kg for Form 1 in male SD rats

| Parameters | 201 | 202 | 203 | Average | SD |
|---|---|---|---|---|---|
| HL_Lambda_z (T_{1/2}, hr) | 3.15 | 3.21 | 3.13 | 3.16 | 0.04 |
| Tₘₐₓ (hr) | 6.00 | 6.00 | 6.00 | 6.00 | 0.00 |
| Cₘₐₓ (ng/mL) | 6026.6 | 7399.7 | 6019.1 | 6481.8 | 794.9 |
| AUCₗₐₛₜ (hr*ng/ml) | 71521.5 | 87858.7 | 67343.3 | 75574.5 | 10841.6 |
| AUC_{INF}_{_{—}}_{pred} (hr*ng/ml) | 72081.3 | 88590.9 | 67845.1 | 76172.4 | 10961.3 |
| MRTₗₐₛₜ (hr) | 6.64 | 6.51 | 6.37 | 6.51 | 0.13 |
| V_{z}_{_{—}}_{F}_{_{—}}_{pred} (L/kg) | 0.63 | 0.52 | 0.67 | 0.61 | 0.07 |
| Cl_{_{—}}_{F}_{_{—}}_{pred} (L/hr/kg) | 0.14 | 0.11 | 0.15 | 0.13 | 0.02 |
| λz Calculation Time Range (hr) | 6-24 | 6-24 | 6-24 | NA | NA |
| F(%) | 41.21 | 50.62 | 38.80 | 43.54 | 6.25 |

Note:
F(%) was calculated by intravenous administration of 1 mg/kg

TABLE 10
Pharmacokinetic parameters of noncompartmental model following
oral administration of 10 mg/kg for Form 2 in male SD rats

| Parameters | 301 | 302 | 303 | Average | SD |
|---|---|---|---|---|---|
| HL_Lambda_z (T_{1/2}, hr) | 2.90 | 3.32 | 3.01 | 3.08 | 0.22 |
| Tₘₐₓ (hr) | 4.00 | 6.00 | 2.00 | 4.00 | 2.00 |
| Cₘₐₓ (ng/mL) | 8489.9 | 8030.2 | 7328.5 | 7949.5 | 584.9 |
| AUCₗₐₛₜ (hr*ng/ml) | 91923.1 | 108067.6 | 65456.4 | 88482.4 | 21513.0 |
| AUC_{INF}_{_{—}}_{pred} (hr*ng/ml) | 92375.2 | 109095.0 | 65798.9 | 89089.7 | 21834.3 |
| MRTₗₐₛₜ (hr) | 6.15 | 6.66 | 5.56 | 6.12 | 0.55 |
| V_{z}_{_{—}}_{F}_{_{—}}_{pred} (L/kg) | 0.45 | 0.44 | 0.66 | 0.52 | 0.12 |
| Cl_{_{—}}_{F}_{_{—}}_{pred} (L/hr/kg) | 0.11 | 0.09 | 0.15 | 0.12 | 0.03 |
| λz Calculation Time Range (hr) | 6-24 | 6-24 | 6-24 | NA | NA |
| F(%) | 52.96 | 62.26 | 37.71 | 50.98 | 12.39 |

Note:
F(%) was calculated by intravenous administration of 1 mg/kg

TABLE 11
Pharmacokinetic parameters of noncompartmental model following
oral administration of 10 mg/kg for Form 5Din male SD rats

| Parameters | 401 | 402 | 403 | Average | SD |
|---|---|---|---|---|---|
| HL_Lambda_z (T_{1/2}, hr) | 3.55 | 3.44 | 3.20 | 3.40 | 0.18 |
| Tₘₐₓ (hr) | 4.00 | 2.00 | 4.00 | 3.33 | 1.15 |
| Cₘₐₓ (ng/mL) | 8543.5 | 8879.3 | 9523.3 | 8982.0 | 497.9 |
| AUCₗₐₛₜ (hr*ng/ml) | 95560.7 | 84721.7 | 95462.1 | 91914.8 | 6229.6 |
| AUC_{INF}_{_{—}}_{pred} (hr*ng/ml) | 96578.2 | 85492.7 | 96111.1 | 92727.3 | 6269.7 |
| MRTₗₐₛₜ (hr) | 6.11 | 5.81 | 5.95 | 5.95 | 0.15 |
| V_{z}_{_{—}}_{F}_{_{—}}_{pred} (L/kg) | 0.53 | 0.58 | 0.48 | 0.53 | 0.05 |
| Cl_{_{—}}_{F}_{_{—}}_{pred} (L/hr/kg) | 0.10 | 0.12 | 0.10 | 0.11 | 0.01 |
| λz Calculation Time Range (hr) | 6-24 | 6-24 | 6-24 | NA | NA |
| F(%) | 55.06 | 48.81 | 55.00 | 52.96 | 3.59 |

Note:
F(%) was calculated by intravenous administration of 1 mg/kg

### Analysis of Pharmacokinetic Results on SD Rat

The pharmacokinetic results showed that Form 2 had higher exposure (AUC₀₋₁) and better bioavailability than Form 1 and may be preferable to Form 1. Following oral administration, the exposure (AUC₀₋₁) and bioavailability of Form 2 were approximately the same as Form 5D. The exposure for Form 5D was approximately the same as results from third parties. Comparing results following intravenous administration in SD rats at 1 mg/kg and at 5 mg/kg dosing levels, the exposure of the latter was about 5 times of the former. Meanwhile, the bioavailability at both dosing levels were calculated to be about 50%. Both Form 1 and Form 2 have sufficiently high bioavailability and are considered to be suitable for development into oral formulations.

### Example 10: Stability Studies

The stability of Compound I Form 2 was analyzed by the following two experiments.

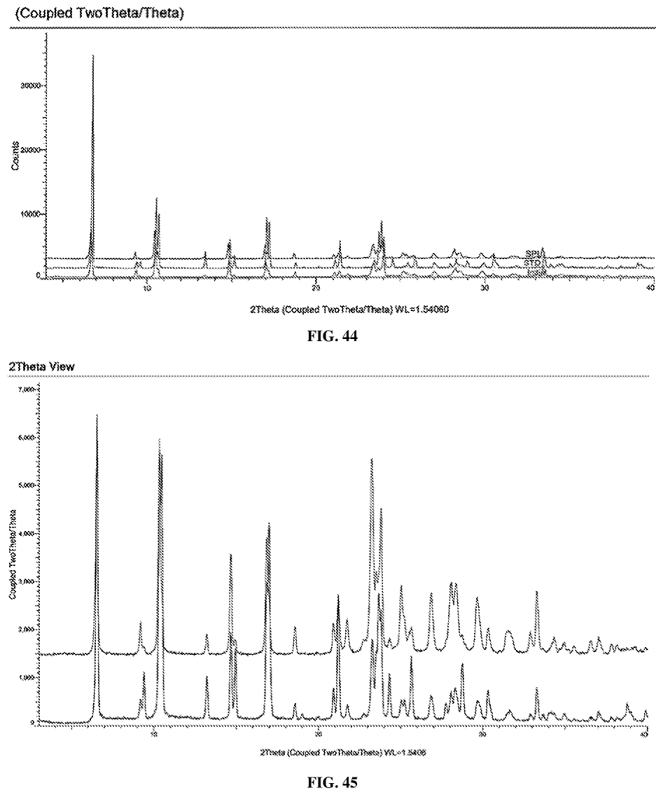

Experiment A: The following is an X-ray Powder Diffraction Analytical method used to obtain the data shown in FIG. 43 and FIG. 44, and which complies with ChP<0451>/USP/NF<941>/EP10.6 2.9.33. Standard XRPD patterns were collected using a Bruker D8 Advance diffractometer or equivalent instrument.

Parameters for collecting the XRPD shown in FIG. 43 and FIG. 44 is as follows:

| | Cu, k-Alpha1, | | |
|---|---|---|---|
| | (λ = | | |
| X-ray Generator | 1.54060 \acute{Å}) | Tube Voltage (kV) | 40 |
| Tube Current (mA) | 40 | Divergence Slit (mm) | 0.6 |
| Primary Soller Slit | 2.5 | Secondary Soller Slit | 2.5 |
| (deg) | | (deg) | |
| Scan axis | 2-Theta/Theta | Step size(deg) | 0.02 |
| Scanning Scope (deg) | 4-40 | Time/step(s) | 0.12 |

| | |
|---|---|
| Scan mode | Continuous PSD fast |

FIG. 43 shows a comparative X-ray powder diffraction (XRPD) plot between a Compound I Form 2 sample placed under conditions of 25° C.±2° C./60% RH±5% RH for 12 months, the Compound I Form 2 sample at 0 days, and a Compound I Form 2 reference standard. In FIG. 43, the top curve represents the Compound I Form 2 sample placed under conditions of 25° C.=2° C./60% RH±5% RH for 12 months (SPL), the middle curve represents the Compound I Form 2 sample placed for 0 days (initial), and the bottom curve represents the Compound I Form 2 reference standard (STD).

FIG. 44 presents a comparative XRPD plot between a Compound I Form 2 sample placed under conditions of 40° C.±2° C./75% RH±5% RH for 6 months, a Compound I Form 2 sample placed for 0 days, and a Compound I Form 2 reference standard. In FIG. 44, the top curve represents the Compound I Form 2 sample placed under conditions of 40° C.±2° C./75% RH±5% RH for 6 months (SPL), the bottom curve represents the Compound I Form 2 sample at 0 days (initial), and the middle curve represents a Compound I Form 2 reference standard (STD).

FIG. 43 and FIG. 44 show that the XRPD patterns of Compound I Form 2 remain consistent with the Form 2 reference standard, both when initially prepared (0 days) and when placed under long-term conditions of 25° C.=2° C./60% RH±5% RH and 40° C.±2° C./75% RH±5% RH. This indicates that Compound I Form 2 remains stable over a period of at least 12 months at 25° C. and at least 6 months at 40° C.

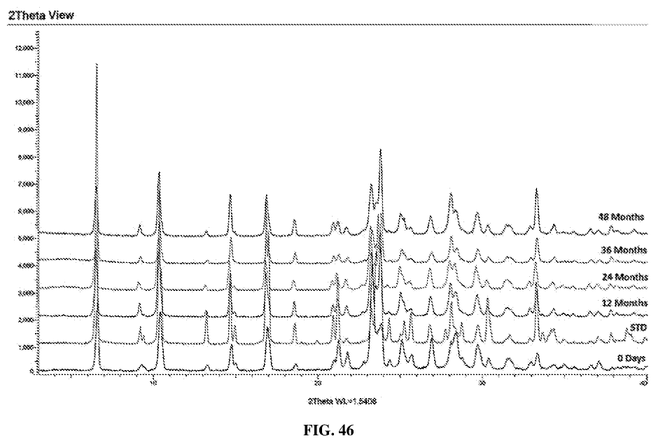

Experiment B: The following is an X-ray Powder Diffraction analytical method used to obtain the data shown in FIG. 45 and FIG. 46. The XRPD patterns shown in FIG. 45 and FIG. 46 were collected using a Bruker D2 Phaser X-ray diffractometer. Detector: PSD LynxEye; Sample holder: Zero background sample holder or equivalent; Software: DIFFRAC. Measurement Center, Version V6.5.0 or equivalent; Diffractometer Settings: Diffractometer type: 02 phaser; Goniometer Type: Theta/Theta; Sample Stage: Standard rotating stage; Goniometer Diameter: 282.2 mm; Divergence slit: 1.0 mm; Primary seller slit: 2.5°; Secondary seller slit: 2.5°; Airscatter screen module: 1.0 mm; Tube Element: Cu; Tube parameters: voltage 30 kV; current 10 mA; Scan Parameters: SSD160: Locked coupled; SSD160-2: coupled Two Theta/Theta Scan Type: SSD160: Locked coupled; SSD160-2: coupled Two Theta/Theta; Continuous PSD fast Scan Mode: Continuous PSD fast scan; Rotation Speed: 20 rpm; Start: 3° ˜40° (2 0); Scan Step: 0.02° (2 0); Scan Speed: 0.2s/step; Detector opening: 4.5°; Sample Analysis; Test the sample with the Diffractometer Settings and Scan Parameters. The powder X-ray diffraction pattern of the sample was recorded and calculations were performed according to Bruker D2 Phaser X-ray Diffractometer User Manual V6-Identification of Crystal Form by X-Ray Powder Diffraction (XRPD)-Bruker D2 Phaser.

FIG. 45 shows a comparative XRPD plot between a Compound I Form 2 sample placed under conditions of 40° C.±2° C./75% RH±5% RH for 6 months and a Compound I Form 2 reference standard. In FIG. 45, the bottom curve represents a Compound I Form 2 reference standard, and the top curve represents the Compound I Form 2 sample placed under conditions of 40° C.±2° C./75% RH±5% RH for 6 months.

FIG. 46 presents a comparative XRPD plot between the Compound I Form 2 sample placed under conditions of 25° C.±2° C./60% RH±5% RH for 12 months, 24 months, 36 months, and 48 months, and the Compound I Form 2 reference standard. Additionally, the bottom curve represents the Form 2 sample at 0 days, while the subsequent curves above represent a Compound I Form 2 reference standard (STD), and the Compound I Form 2 sample placed under conditions of 25° C.±2° C./60% RH±5% RH for 12 months, 24 months, 36 months, and 48 months.

FIG. 45 and FIG. 46 show that the XRPD patterns of Compound I Form 2 remain consistent with the Compound I Form 2 reference standard, both when initially prepared (0 days) and when placed under long-term conditions of 40° C.±2° C./75% RH±5% RH for 6 months and 25° C.±2° C./60% RH±5% RH for 48 months. This indicates that Compound I Form 2 remains stable over a period of at least 6 months at 40° C. and at least 48 months at 25° C.

All patents and other references cited in the specification are indicative of the level of skill of those skilled in the art to which the disclosure pertains, and are incorporated by reference in their entireties, including any tables and figures, to the same extent as if each reference had been incorporated by reference in its entirety individually.

One skilled in the art would readily appreciate that the present disclosure is well adapted to obtain the ends and advantages mentioned, as well as those inherent therein. The methods, variances, and compositions described herein as presently representative of preferred embodiments are exemplary and are not intended as limitations on the scope of the disclosure. Changes therein and other uses will occur to those skilled in the art, which are encompassed within the spirit of the disclosure, are defined by the scope of the claims.

The disclosure illustratively described herein suitably may be practiced in the absence of any element or elements, limitation or limitations which is not specifically disclosed herein. Thus, for example, in each instance herein any of the terms “comprising”, “consisting essentially of” and “consisting of” may be replaced with either of the other two terms. Thus, for an embodiment of the disclosure using one of the terms, the disclosure also includes another embodiment wherein one of these terms is replaced with another of these terms. In each embodiment, the terms have their established meaning. Thus, for example, one embodiment may encompass a method “comprising” a series of steps, another embodiment would encompass a method “consisting essentially of” the same steps, and a third embodiment would encompass a method “consisting of” the same steps. The terms and expressions which have been employed are used as terms of description and not of limitation, and there is no intention that in the use of such terms and expressions of excluding any equivalents of the features shown and described or portions thereof, but it is recognized that various modifications are possible within the scope of the disclosure claimed. Thus, it should be understood that although the present disclosure has been specifically disclosed by preferred embodiments and optional features, modification and variation of the concepts herein disclosed may be resorted to by those skilled in the art, and that such modifications and variations are considered to be within the scope of this disclosure as defined by the appended claims.

In addition, where features or aspects of the disclosure are described in terms of Markush groups or other grouping of alternatives, those skilled in the art will recognize that the disclosure is also thereby described in terms of any individual member or subgroup of members of the Markush group or other group.

Also, unless indicated to the contrary, where various numerical values are provided for embodiments, additional embodiments are described by taking any 2 different values as the endpoints of a range. Such ranges are also within the scope of the described disclosure.

Thus, additional embodiments are within the scope of the disclosure and within the following claims.

What is claimed is:

1. A crystalline form of Compound I: 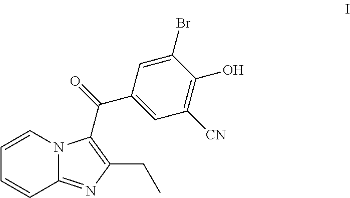 [SMILES: CCc1nc2ccccn2c1C(=O)c1cc(Br)c(O)c(C#N)c1]
or a crystalline form of a pharmaceutically acceptable salt or solvate of Compound I.

2. The crystalline form according to claim 1, which is Compound I Form 1, characterized by an X-ray powder diffractogram comprising peaks (±0.2°) at 15.0, 22.6, 25.8, 32.0, and 41.3° 2θ as determined on a diffractometer using Cu-Kα radiation.

3. The crystalline form according to claim 2, further characterized by:
i) the X-Ray powder diffractogram comprising further peaks at 25.5, 27.1, 27.5, and 28.3°2θ±0.2°;
ii) a diffractogram substantially as shown in FIG. 1;
iii) a differential scanning calorimetry (DSC) comprising endotherms peaking at about 102° C. and about 158° C.; or
iv) a thermogravimetric analysis (TGA) comprising a thermogram substantially as shown in FIG. 2.

4. The crystalline form according to claim 1, which is Compound I Form 2, characterized by an X-ray powder diffractogram comprising peaks (±0.2°) at 6.7, 10.5, 17.0, 23.4, and 26.9° 2θ as determined on a diffractometer using Cu-Kα radiation.

5. The crystalline form according to claim 4, further characterized by:
i) the X-Ray powder diffractogram comprising further peaks at 14.8, 21.3, 28.4, and 29.8° 2θ+0.2°;
ii) a diffractogram comprising peaks substantially as shown in FIG. 4;
iii) a differential scanning calorimetry (DSC) comprising an endotherm peaking at about 256° C.; or
iv) a thermogravimetric analysis (TGA) comprising a thermogram substantially as shown in FIG. 5.

6. The crystalline form according to claim 1, which is Compound I Form 3, characterized by an X-ray powder diffractogram comprising peaks (±0.2°) at 16.2, 23.1, 28.0, and 31.8° 2θ as determined on a diffractometer using Cu-Kα radiation.

7. The crystalline form according to claim 6, further characterized by:
i) the X-Ray powder diffractogram comprising further peaks at 13.0, 14.5, 17.1, 19.6, 22.8, 24.1, 26.5, 26.9, 27.3, 30.1, and 30.5° 2θ±0.2°;
ii) a diffractogram substantially as shown in FIG. 7;
iii) a differential scanning calorimetry (DSC) comprising endotherms peaking at about 171° C. and 251° C.; or
iv) a thermogravimetric analysis (TGA) comprising a thermogram substantially as shown in FIG. 8.

8. The crystalline form according to claim 1, which is Compound I Form 4, characterized by an X-ray powder diffractogram comprising peaks (±0.2°) at 11.2, 22.4, 25.0, 27.4, and 29.1° 2θ as determined on a diffractometer using Cu-Kα radiation.

9. The crystalline form according to claim 8, further characterized by:
i) the X-Ray powder diffractogram comprising further peaks at 17.2, 22.2, 23.7, 24.1, 27.1, and 30.7° 2θ ±0.2°;
ii) a diffractogram substantially as shown in FIG. 10;
iii) a differential scanning calorimetry (DSC) comprising endotherms peaking at about 102° C., 149° C., and 256° C.; or
iv) a thermogravimetric analysis (TGA) comprising a thermogram substantially as shown in FIG. 11.

10. The crystalline form according to claim 1, which is Compound I Form 5D, characterized by an X-ray powder diffractogram comprising peaks (±0.2°) at 12.6, 14.4, 15.9, 22.0, 23.0, 27.0, 27.7, and 29.6 °2θ as determined on a diffractometer using Cu-Kα radiation.

11. The crystalline form according to claim 10, further characterized by:
i) an X-Ray powder diffractogram comprising further peaks at 9.6, 11.4, 16.7, 18.1, 19.1, 20.3, 24.0, 25.4, 28.6, 30.1, 31.7, 32.4, and 33.5° 2θ±0.2°;
ii) a diffractogram substantially as shown in FIG. 13D;
iii) a differential scanning calorimetry (DSC) comprising endotherms peaking at about 197° C.; or
iv) a thermogravimetric analysis (TGA) comprising a thermogram substantially as shown in FIG. 14D.

12. The crystalline form according to claim 1, which is Compound I Form 6 and is a crystalline form of a partial acetic acid solvate of the Compound I, wherein the Compound I Form 6 is characterized by an X-ray powder diffractogram comprising peaks (±0.2°) at 15.9, 20.6, 24.2, 24.5, 25.8, 26.8, and 30.4° 2θ as determined on a diffractometer using Cu-Kα radiation.

13. The crystalline form according to claim 12, further characterized by:
i) an X-Ray powder diffractogram comprising further peaks at 9.1, 14.8, 15.5, 17.3, 19.8, 23.5, 26.1, 28.0, 28.4, 31.7, and 36.3° 2θ±0.2°;
ii) a diffractogram comprising peaks substantially as shown in FIG. 16;
iii) a differential scanning calorimetry (DSC) comprising an endotherm peaking at about 151° C. and 260° C.; or
iv) a thermogravimetric analysis (TGA) comprising a thermogram substantially as shown in FIG. 17.

14. A composition comprising two or more compounds selected from the group consisting of Compound I Form 1 according to claim 2, Compound I Form 2 according to claim 4, Compound I Form 3 according to claim 6, Compound I Form 4 according to claim 8, Compound I Form 5D according to claim 10, and Compound I Form 6 according to claim 12.

15. The composition of claim 14, wherein the composition comprises Compound I Form 1 according to claim 2 and Compound I Form 2 according to claim 4.

16. The composition of claim 15, wherein the composition comprises at least 50% w/w of Compound I Form 2.

17. A composition comprising Compound I, wherein at least 85%, or at least 85%, or at least 90%, or at least 95%, or at least 97%, or at least 98%, or at least 99%, or at least 99.5%, of the Compound I in the composition is present as Compound I Form 2.

18. A pharmaceutical composition comprising a compound selected from the group consisting of Compound I Form 1 according to claim 2, Compound I Form 2 according to claim 4, Compound I Form 3 according to claim 6, Compound I Form 4 according to claim 8, Compound I Form 5D according to claim 10, and Compound I Form 6 according to claim 12, and further comprising a pharmaceutically acceptable excipient.

19. A method for treating a subject suffering from or at risk of a URAT1-mediated disease or condition, said method comprising administering to said subject an effective amount of Compound I Form 1 according to claim 2, Compound I Form 2 according to claim 4, Compound I Form 3 according to claim 6, Compound I Form 4 according to claim 8, Compound I Form SD according to claim 10, and Compound I Form 6 according to claim 12, and a pharmaceutically acceptable excipient, the composition according to claim 17, or the pharmaceutical composition according to claim 18.

20. The method of claim 19, wherein the diseases or conditions are associated with insufficient renal elimination of uric acid.

21. The method of claim 19, wherein the disease or condition is gout or hyperuricemia.
